(19) United States
(12) Patent Application Publication
(10) Pub. No.: US 2008/0058629 A1
(43) Pub. Date: Mar. 6, 2008

(54) OPTICAL FIBER SCOPE WITH BOTH NON-RESONANT ILLUMINATION AND RESONANT COLLECTION/IMAGING FOR MULTIPLE MODES OF OPERATION

(75) Inventors: Eric Seibel, Seattle, WA (US); Richard S. Johnston, Sammamish, WA (US); Timothy Soper, Seattle, WA (US); Charles D. Melville, Issaquah, WA (US)

Correspondence Address: LAW OFFICES OF RONALD M ANDERSON, 600 108TH AVE, NE, SUITE 507, BELLEVUE, WA, 98004, US

(73) Assignee: University of washington, Seattle, WA (US)

(21) Appl. No.: 11/465,988

(22) Filed: Aug. 21, 2006

(51) Int. Cl. A61B 5/00

(52) U.S. Cl. 600/368

(57) ABSTRACT

A scanning fiber endoscope (SFE) system selectively operable in a plurality of different modes. One or more illumination optical fibers convey different types of light to an internal site. A scanner that is resonantly driven in a desired pattern collects light from the internal site. The scanner can be a cantilevered distal end of a scanning optical fiber or a scanning mirror. The illumination optical fiber(s) can be moved in a non-resonant manner to alter the direction at which the light is emitted. In a therapy mode, a relatively high-power light can be applied to the site, while in a monitoring mode, the scanner can be used to image the tissue at the internal site after or during therapy. Exemplary SFE probes are disclosed for measuring scattering angle (which can detect larger cancer cell nuclear-to-cytoplasmic ratio), absorption depth, axial distance to tissue, and other conditions at the internal site.

### GOVERNMENT RIGHTS

This invention was funded at least in part with a grant (No. R21/R33 CA094303) from the National Institutes of Health (NIH), and the U.S. government may have certain rights in this invention.

### BACKGROUND

In minimally-invasive medical procedures, such as procedures performed with an endoscope, an optical therapy is typically provided by a laser that is connected to a single large-core multimode optical fiber. The distal end of this multimode optical fiber is introduced into the body by hand and is advanced so that its tip physically contacts the tissue of interest at an internal site. Alternatively, the optical beam emerging from the distal tip of the optical fiber is scanned by hand onto the tissue, or an optical diffuser attached to the distal end of the optical fiber is employed to diffusely spread the optical radiation dose across the tissue of interest. Imaging the tissue of interest before and after the therapy (and monitoring the efficacy or extent of the therapy) is typically done using a flexible or rigid endoscope that channels a monitoring optical fiber to the region of interest within the body. In cardiovascular applications, an optical fiber is commonly introduced with a non-imaging catheter, and imaging of the procedure is done externally to the patient's body, using such conventional imaging methods, such as fluoroscopy.

Recently, new ultra-thin and flexible embodiments of a scanning fiber endoscope (SFE) or CatheterScope technology have been developed that enable optical radiation to be introduced into the body in devices used for minimally invasive medical procedures, such as flexible endoscopes, rigid laparoscopes, bronchoscopes, and other rigid or flexible scopes, as well as introduction via catheters. The optical radiation can be delivered to the tissue in one of several ways, such as by being coupled directly to a resonant fiber scanner core and/or cladding, or can be delivered using single or dual or multi cladding optical fibers, or delivered via an optical fiber in the more traditional way, but in parallel with a separate resonant optical fiber scanner.

A previously developed SFE technology uses a resonant optical fiber scanner to deliver light to a site, and in this approach, the light spot is always moving across the plane of illumination. If a large therapeutic dose of optical radiation is required, then an extremely high-power laser must turn on and off for the instant in time when the scanning fiber tip is aligned with the required illumination pixels that correspond to the region of interest where therapy is to be delivered. The previously proposed direct integration of the optical therapy with the SFE imaging is therefore limited in the level of optical power that can be delivered in this short interval of time. Overcoming this limitation can be costly.

Clearly, it would be desirable to monitor an internal site in real-time, while therapy is being delivered (or while other procedures are being carried out), without limiting the duration of the therapy (or the other procedures) to the alignment time for a scanning optical fiber. For example, it would also be desirable to facilitate thermal or fluorescence monitoring of the progression of optical therapy, so that an image of the site using these wavelengths can be displayed in real-time to the medical practitioner.

### SUMMARY

Various exemplary embodiments of scanning probes have been developed for use in carrying out a number of different functions within a patient's body, by selectively operating in a plurality of different modes that use a plurality of different types of light. Specifically, certain embodiments of the probe are useful for implementing at least two different modes. For example, the different modes can include imaging a site using visible light, performing diagnosis of medical conditions at the site using light that is useful in measuring condition of the tissue at the site, rendering therapy to the site with relatively high-power light of appropriate waveband, and monitoring the site to determine the results of therapy rendered—for example, in real-time. In connection with diagnosis of a medical condition, one embodiment of an SFE probe has been developed that uses a resonant scanner to image an internal site or collect light from the site. The SFE probe is able to determine a scattering angle of light from the site, and this information can be used, for example, in diagnosing cancer, based upon the size of cell nuclei determined as a function of the scattering angle measured and the frequency or wavelength of the light. Absorption of light by the tissue relative to depth of light penetration (at a desired wavelength) can be determined, since the absorption is proportional to optical path length, which can be varied by the radius of a spiral scan provided by the scanner in the SFE probe from a point or beam of illumination. Further, an axial length between a probe and a surface of tissue can be determined using the scanner in an exemplary SFE probe to collect light reflected from the illuminated tissue. These functions and applications of various embodiments of exemplary novel SFE probes to implement at least two of these different modes, using a fixed or non-resonant movable illumination optical fiber, and a resonant scanner for imaging or collecting light, are described in detail below.

Specifically, an exemplary optical fiber system for illuminating an internal site within a body of a patient with different types of light in different modes and responding to light received from the internal site is described below. The system includes a plurality of light sources that produce the different types of light. An illumination optical fiber having a distal end is selectively coupled to the plurality of light sources to convey the light for a current mode to the distal end of the illumination optical fiber, to illuminate the internal site. A scanning driver or actuator adapted to be energized by a drive signal is included and is connected to the scanning optical fiber adjacent to its distal end. The scanning driver or actuator is configured to drive a scanner so as to scan at least a portion of the internal site in a desired pattern. Light received from at least a portion of the internal site that has been illuminated by the light from the illumination optical fiber in the current mode enters the distal end of the scanner optical fiber and is conveyed by the scanner optical fiber toward its proximal end. A light sensor or detector is coupled to the scanner optical fiber to receive the light that it conveys. In response, the light sensor produces an output signal indicative of at least one parameter of the light received from the internal site.

Another aspect of the approach is directed to a method for scanning an internal site within a patient's body in a plurality of different modes. For each of at least two modes of the plurality of different modes that is implemented, the method includes the steps of conveying one of a plurality of different types of light from a source selected for use in a current mode of the at least two modes, toward a distal end of an illumination optical fiber. The light emitted from the distal end of the illumination optical fiber is directed onto the internal site; the distal end of the illumination optical fiber can be stationary or relatively slowly movable with a non-resonant motion. At least a portion of the internal site is scanned to collect received light. The received light from at least the portion of the internal site is conveyed through a scanner optical fiber and toward a proximal end of the scanner optical fiber. The received light is detected, producing an output signal in response thereto. At least one parameter of at least a portion of the internal site is determined using the output signal, for the current mode that is then being implemented.

Yet another aspect is directed to an optical fiber scope for illuminating an internal site within a body of a patient with a plurality of different types of light during a plurality of different modes, and responding to light received from the internal site. In one exemplary configuration, the optical fiber scope includes a plurality of different light sources producing different types of light for illuminating the internal site. An elongate housing is disposed at a distal end of the optical fiber scope. And, a scanner is disposed generally centrally at the distal end of the optical fiber scope. The scanner is driven to move in a desired pattern at approximately a resonant frequency and configured so that received light from at least a desired portion of the internal site is collected by the scanner and conveyed through a scanner optical fiber toward a proximal end of the scanner optical fiber. A plurality of illuminating optical fibers having distal ends are spaced apart and disposed around the scanner, within the elongate housing. The plurality of illuminating optical fibers convey light from a selected one of the plurality of different light sources toward the distal ends of the illuminating optical fibers during operation in a current mode. The light emitted from the distal ends of the illuminating optical fibers is then directed to the internal site. A sensor is coupled to the scanner optical fiber and is responsive to the received light that is conveyed through the scanner optical fiber. The sensor produces an output signal that can be processed to provide data relating to tissue at the internal site for the current mode that is being implemented by the optical fiber scope.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1 is a schematic elevational view of a distal end of an exemplary probe having non-resonant illumination optical fibers and a resonant scanning optical fiber that is collecting light and/or imaging an internal site in a patient's body;

FIG. 2 is a schematic elevational view of a distal end of another exemplary probe having non-resonant illumination optical fibers and a resonant scanning mirror that is collecting light and/or imaging an internal site in a patient's body;

FIG. 3 is a schematic elevational view of a distal end of yet another exemplary probe that is adapted for carrying out relatively high-power laser therapy using ultra-fast laser pulses and monitoring a site by imaging using non-confocal or pseudo-confocal multi-photon scanning of an internal site using a resonant optical fiber scanner;

FIGS. 4A-4E respectively illustrate a spiral scan pattern, a linear scanning pattern that rotates about a central axis, a raster scanning pattern, a propeller scan pattern that rotates about a central axis, and a Lissajous scan pattern, as examples of some of the different resonant scanning patterns that can be employed;

FIG. 5 schematically illustrates a distal end of an exemplary probe, showing a plurality of optical fibers that are used for different purposes grouped around a resonant scanner;

FIG. 6 schematically illustrates another exemplary embodiment for a probe having non-resonant illumination optical fibers on opposite sides of a scanning optical fiber, and using a plurality of lenses for focusing on an internal site;

FIG. 7 is a schematic block diagram of a scanning system that employs a probe in accord with one exemplary embodiment;

FIG. 8 is a schematic diagram illustrating the relationship between specular and diffuse light reflecting from a tissue surface;

FIG. 9 is a controller for the scanning system that is usable, for example, to determine a distance between a distal portion of a probe and a surface;

FIG. 10 is a schematic illustration showing the functional relationship between illumination optical fibers and a scanner relative to an internal site surface;

FIGS. 11A-11C illustrate three different specular reflection patterns at different distances between the distal end of a probe and the surface of internal tissue;

FIGS. 11D-11E illustrate two different illumination patterns at the same distance between the distal end of a probe and the surface of internal tissue when the probe is rotated during illumination and when illumination is displaced radially while probe is being rotated;

FIG. 12 illustrates an exemplary scanning device having a resonant scanning optical fiber that can be driven to scan a region in any of a plurality of different scan patterns, including a variable radius circular, or a spiral scanning mode, and when imaging the region, collects light from the region that is illuminated by light from a plurality of illumination optical fibers disposed around a lens assembly at the distal end of the scanning device;

FIG. 13 is a schematic cross-sectional view of one exemplary embodiment that uses a fluid-filled balloon to move illumination optical fibers (non-resonantly) to selectively change where light emitted therefrom is incident on an internal surface;

FIG. 14 is a schematic cross-sectional view of one side of another exemplary embodiment that uses bimorph displacers to move illumination optical fibers to selectively change where light emitted therefrom is incident on an internal surface;

FIGS. 15A-15B are schematic sectional isometric views of a distal portion (one of the four illumination optical fibers has been cut away to simplify the view) of an exemplary embodiment similar to that of FIG. 13, respectively illustrating positions of the illumination fibers when a balloon is generally not inflated, and alternative positions of the illuminating optical fibers after a pressurized fluid has at least partially inflated the balloon to a greater extent;

FIG. 15C-15D are schematic isometric views of a distal portion of the exemplary embodiment of FIGS. 15A and 15B, respectively illustrating positions of the illumination fibers when the balloon is generally not inflated, and the alternative positions of the illuminating optical fibers when fluid is used to at least partially inflate the balloon;

FIG. 16 is a schematic cross-sectional view of one side of a distal portion of yet another exemplary embodiment of an exemplary SFE probe, wherein a cable or line extending proximally through a lumen is used to move an illumination optical fiber to selectively change where light emitted therefrom is incident on an internal surface;

FIG. 17 is a schematic cross-sectional view of an exemplary SFE probe in which a Fourier transform lens is employed to determine scattering and thus, the size of a cellular component or particle that has scattered the light;

FIG. 18 illustrates a cross-sectional view of still another exemplary embodiment of a probe that enables both high resolution imaging and capturing different scattering angles, using a Fourier transform lens and a longitudinally movable, resonant optical fiber scanner;

FIG. 19 is a cross-sectional view of a probe that includes illumination optical fibers coupled to light sources of different characteristics, e.g., for measuring/mapping local diffuse reflections collected in an inner cladding of a scanning optical fiber, as well as high resolution imaging using a singlemode core of the scanning optical fiber;

FIG. 20 is a schematic view of an exemplary embodiment of an exemplary SFE probe (having a scanner—not visible) that includes the two illumination optical fibers of FIG. 19, and 12 other multimode optical fibers that can be used for various functions;

FIG. 21 is a schematic axial view of an internal site that is illuminated with light from the two illumination optical fibers, illustrating vectors for the light emitted from each different illumination optical fiber, which has different properties; and

FIG. 22 is an exemplary functional block diagram illustrating the functional components and signal flow in a system that employs an SFE probe such as those described herein, for any one or more of imaging, monitoring, rendering diagnoses, and providing therapy to an internal site in a patient's body.

### DESCRIPTION

### Figures and Disclosed Embodiments are not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein.

### Exemplary Embodiments of Resonant Scanners

Each of the plurality of exemplary embodiments disclosed herein is usable in a system for selectively implementing at least two different modes using different types of light. For example, the plurality of different modes include: (1) a diagnostic mode used to determine a condition of tissue at an internal site by responding to light received from the tissue to determine parameters indicative of the tissue condition; (2) an imaging mode used to produce an image of the internal site using imaging (e.g., using visible light—monochromatic or light with red, green, and blue (RGB) components); (3) a therapy mode for administering therapy to tissue at the internal site using a relatively high-power therapy light; and, (4) a monitoring mode, which is used to assess the condition of tissue at the internal site before, after, and/or during administration of therapy to the internal site, by imaging the site. The modes implemented by each exemplary embodiment discussed below are not always specifically indicated, but will be apparent to those of ordinary skill in the art, based upon the disclosure.

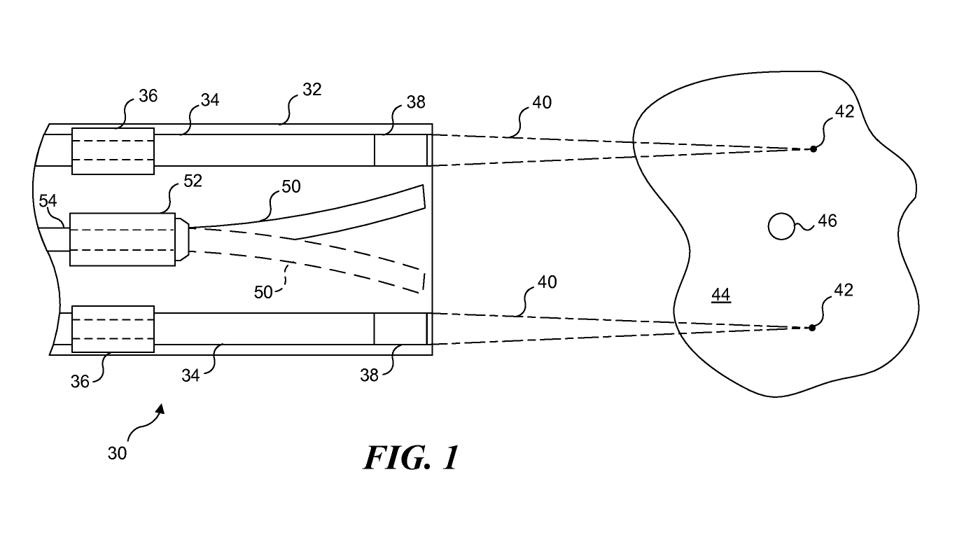

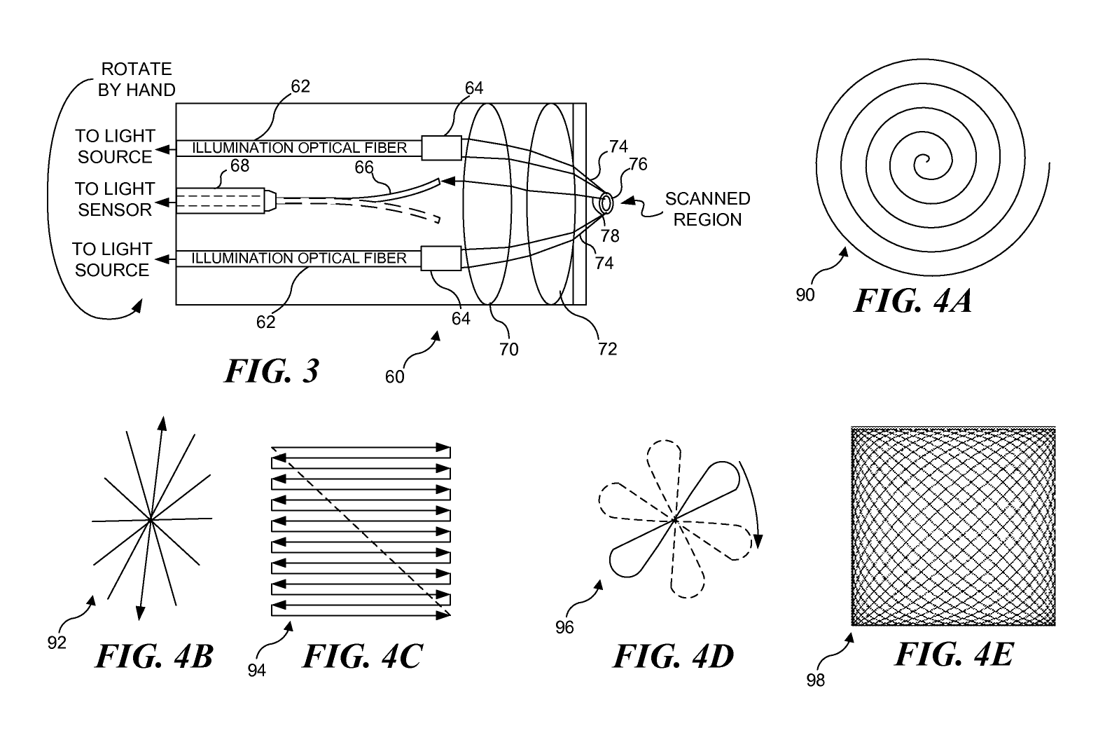

In several of the exemplary embodiments illustrated in the Figures and discussed below, such as the embodiments of FIGS. 1, 2, and 10, an objective lens system is not shown. However, for probes that are used in endoscopy applications of this technology, it is contemplated that one of many possible different combinations of objective lens system would likely be included for use, both to provide high magnification focus (and imaging), and to ensure high light collection efficiency, using large numerical aperture optics. Exemplary objective lens systems that might be included are shown in FIGS. 3, 6, 12, 13, and 17-19. For endoscopic applications of the illustrated exemplary probes, such an imaging lens system might be disposed between the resonant scanner and the illuminated tissue, to collect light at greater efficiencies using the increased numerical aperture optics. Furthermore, the objective lens system can be designed for tissue contact and/or water immersion at the distal (i.e., front) surface thereof.

A first exemplary embodiment of a probe 30 that is suitable for using a resonant scanner to collect light or image an internal site 44 within the body of a patient (not shown) is illustrated in FIG. 1. Probe 30 includes a housing 32, only a distal portion of which is shown in the schematic view. Within housing 32 is disposed a plurality of illumination optical fibers 34. Only two such illumination optical fibers are illustrated in this Figure, but as will be evident from the discussion below, it is also contemplated the SFE probe can include only a single illumination optical fiber, as well as more than two such optical fibers. Optionally, illumination optical fibers 34 each includes a displacer 36, which is illustrated simply as a schematic block in this Figure, and is intended to represent a mechanism for non-resonantly moving the illumination optical fiber so as to change the direction in which it emits illumination light, thereby selectively changing where the illumination light is incident on internal site 44. As a further option, each of the illumination optical fibers can include a rod lens 38, for example, a gradient index (GRIN) lens, to more effectively focus light emitted from the illumination optical fibers on the internal site.

In the example shown, illumination light 40 emitted through rod lenses 38 is incident on the internal site and is focused at points 42. The illumination optical fibers can have relatively large multimode cores and will thus be able to carry relatively high intensity light at appropriate selected wavelengths to achieve various different functions, as described in detail below. In some exemplary embodiments, one or more illumination optical fibers can comprise singlemode optical fibers.

Probe 30 can be employed in a variety of different applications. In an imaging application, the illumination light conveyed through illumination optical fibers 34 and directed to internal site 44 may be simply white light (i.e., with red, green, and blue components) suitable for illuminating the internal surface, or can be monochromatic. If being used to render therapy to the internal site, a therapeutic optical radiation can normally be conveyed through illumination optical fibers 34 at sufficiently high-power and with sufficient pulse width or duration to achieve the desired therapeutic effect, and without damaging illumination optical fibers 34. Further, if displacer 36 is included, instead of illuminating internal site 44 at two fixed spots 42, illumination light 40 can be focused at other spots or to a single spot, such as spot 46, increasing the effective power delivered to the internal site and reducing any coherence effects.

Also included is a scanning optical fiber 50, which is driven to move in a desired pattern at a resonant or near-resonant frequency by a piezoceramic tube actuator 52, or other suitable scanning driver. The scanning optical fiber can selectively be driven in a linear resonance or near-resonant motion, as indicated by the dash line position of optical fiber 50′, or can be driven in a more complex two-dimensional motion, with a driving force applied relative to two orthogonal axes. Several examples of scanning patterns that can be achieved by scanning optical fiber 50 are illustrated in the drawings and discussed below. Scanning optical fiber 50 comprises the distal end of a singlemode (or multimode) optical fiber 54 that extends toward a proximal end (not shown) where the light that enters the scanning optical fiber can be sensed by a sensor or detector, or be used to produce an image on a display, as discussed in greater detail below. Optionally, one or more objective lenses may be disposed between the optical fiber 54 and internal site 44 to provide high-power magnification and greater light collection efficiency with high numerical aperture optics, as shown in other embodiments discussed below. In various applications and embodiments discussed herein, the scanning optical fiber can also alternatively comprise a multi-clad optical fiber or a combination multimode and singlemode optical fiber.

SFE probe 30 is configured and sized so that it can readily be introduced into the body of a patient in a variety of different devices that are used for minimally invasive medical procedures, such as flexible scopes, rigid laparoscopes, Rocco Scopes, and other rigid or flexible scopes, as well as in connection with catheters. Since scanning optical fiber 50 can image internal site 44 independent of the delivery of illumination light 40 that is used for therapy, the scanning optical fiber can be employed for monitoring internal site 44 in real-time to determine the progress of the therapy and its effect on the internal site. Illumination optical fibers 34 that are used for delivering illumination light to render therapy can also be used to collect infrared (1R) light received from the internal site for monitoring the heat generated during the therapy. At the proximal end of the illumination optical fibers, an illumination laser that is used for therapy and produces near-IR wavelength light can be separated from any collected thermal omissions and longer IR wavelengths using a dichroic beam splitter (not shown), such as a long-pass filter disposed at a 45° angle. The therapeutic optical radiation emitted from illumination optical fibers 34 can be focused by rod lenses 38, which would then be specifically designed for the wavelengths of the therapeutic light.

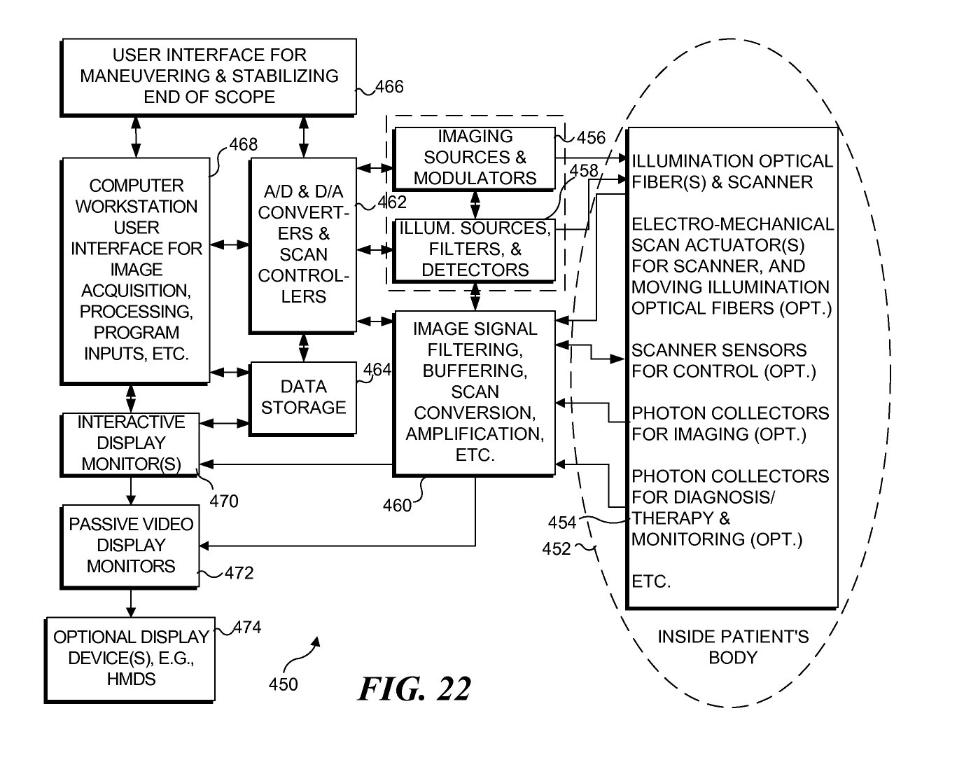

A technique has been developed to register the illumination/therapy plane with the scanning imaging plane for an SFE, such as probe 30. By using a computer for controlling the SFE (see FIG. 22 and the accompanying discussion), it should be possible to accurately register the two image planes. For example, a low-power near-IR light source can be selectively coupled to the proximal ends of illumination optical fibers 34, which are normally used to deliver much higher power light used for therapy. The same sensor or optical detector that is coupled to the proximal end of singlemode (or multimode) optical fiber 54 and normally used for imaging the internal site or for monitoring the results or progress of rendering therapy is then used for mapping the pixel location within a red, green, and blue (RGB) image of the low-power IR spots of light. These spots of measured location, size, and shape will be recorded by the computer system that controls the scanning optical fiber. As desired by the user, a colored circle can be drawn and displayed to the user around each of the spots at full-width-at-half-maximum of the measured optical power distribution during the initialization process. By selectively controlling the SFE probe with the computer system, the SFE probe can be maneuvered so that the imaged region of interest is encircled by one or more of the colored circles before the full higher power therapy light is administered through the illumination optical fibers, thereby ensuring that the therapy is delivered to the desired portion or region of the internal site, in registration with the image of the region provided by the scanning optical fiber.

Depending upon the application to which it is applied, the SFE probe can be designed and configured so that a plurality of beams of illumination light used for optical therapy are combined to a single spot within a imaging field of scanning optical fiber 50 to increase the total intensity of the therapy light that can be delivered to a desired region within scanned surface of internal site 44. Thus, if the therapy light emitted from four illumination optical fibers is focused on a single spot within the imaging field, the total power delivered to the spot will be about four times that of the therapy light delivered from only one of the illumination optical fibers. Alternatively, the plurality of illumination optical fibers can be configured so that the therapy light that they emit forms a square, rectangle, circle, or other shaped area that is about four times the area of a single spot. If a still larger area is desired, the SFE probe can be moved manually, for example to sweep the distal end back and forth or rotate it, so that the optical therapy light is swept over the area of interest at internal site 44. A user interface or display (not shown in FIG. 1, but described in detail below), e.g., of false color, can provide an indication of tissue surface temperature as a real-time feedback of the condition of the tissue while optical therapy is being administered. As a further example, the scanning optical fiber can detect infrared light from the site being administered the optical therapy to provide an indication of the progression of the optical therapy or of the tissue response to the therapy. If heating is not indicative of the progression of optical therapy, scanning optical fiber 50 can instead be configured to image the internal site so as to respond to phosphorescence, auto-fluorescence ratio (wavelength or polarization), or extrinsic fluorescence intensity or emission lifetime (which may be particularly useful if the therapy is photodynamic therapy (PDT)).

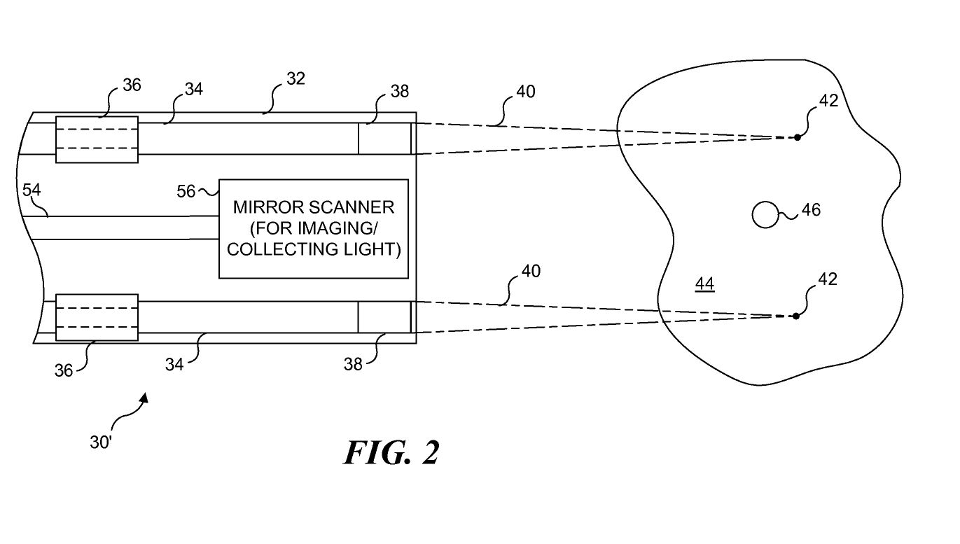

As an alternative to using scanning optical fiber 50 as shown in FIG. 1, an SFE probe 30′ shown in FIG. 2 includes a mirror scanner 56 having one or more reflective surfaces (not shown) driven at a resonant frequency or about a resonant frequency, to scan a desired region, such as spot 46 on internal site 44. Mirror scanner 56 can include one or more moving micro-electro-mechanical system (MEMS) reflective surfaces, or may include one or more piezoelectric or galvanometer drivers coupled to reflective surfaces that are driven in orthogonal directions, e.g., to resonantly scan in the x-direction with one reflective surface and in the y-direction with the other reflective surface. SFE probe 30′ otherwise includes substantially the same components as probe 30, so that illumination light 40 emitted from the distal ends of illumination optical fibers 34 is directed to illuminate internal site 44 at points 42 or as otherwise desired. By properly configuring the illumination fiber(s) to be selectively moved by displacer 36, the illumination optical fiber can direct the illumination light emitted at a desired region or point on internal site 44. Optionally, one or more objective scan lenses may be disposed between mirror scanner 56 and the internal site to provide both high optical magnification with greater numerical aperture, for focusing the illumination and collecting the optical signals.

### Exemplary Embodiment of a Non-Confocal SFE Using High-Power Laser Pulses

To provide both high-power laser illumination to ablate (optically remove) material at the internal site, amplified ultra-short laser pulses are focused onto the surface of the tissue. Typically, these laser amplifiers operate in the near-infrared (NIR) region of the optical spectrum and have pulse durations in the femtosecond (fs) to picosecond (ps) range, although nanosecond (ns) to millisecond (ms) pulses at other optical frequencies may suffice. In order to remove material or ablate tissue at only the focal point and to cause a minimum of collateral damage to other tissue, high pulse repetition rates are avoided. Typically, the light source can comprise an amplified fs-pulsed laser system with an output at about 800 nm wavelength. Laser sources meeting this need are currently available from major USA manufacturers, such as Spectra-Physics, Newport Corporation, and Coherent Inc. To ablate tissue at this NIR optical frequency, pulses at power levels at the tissue of around 1 micro Joule or greater are usually required. To transmit these ultra-fast NIR pulses to the internal site, specifically designed and manufactured photonic crystal optical fibers with hollow cores and/or large-area core diameters have been developed and are available, for example, from Crystal Fibre, Denmark. A singlemode photonics crystal fiber and any rod, GRIN, or objective lens system can be used to focus the high-power laser illumination onto the internal site. By non-resonant scanning of this illumination, single or multiple high-power, ultra-fast pulses can be applied to the tissue surface using low repetition-rate amplifiers. Concurrently, the faster scanning of the internal site by the resonant fiber (or mirror) can be used to collect light signals from the illuminated internal site to monitor the laser cutting process in real-time. The ideal monitoring fiber may be a dual clad optical fiber that allows both high-resolution surface imaging from the singlemode core in addition to multimode light collection within the inner cladding, for monitoring the therapy. Alternatively, a dual clad photonic crystal optical fiber is now available commercially from Crystal Fibre, Denmark. Standard singlemode or multimode optical fibers can be employed, as well as hollow core and band gap optical fibers. The methods of monitoring can include straightforward endoscopic imaging with a scanned beam of visible wavelengths (e.g., red, green, and blue visible light), by collecting the backscattered light from the internal site. Additional methods of monitoring the laser therapy include the mapping of fluorescent light, thermal emissions, and by imaging the tissue with wavelengths of light outside the visible spectrum. Also, tissue optical properties can be assessed concurrently during the therapy or in time series to monitor the disease state of the remaining tissue using both non-resonant fiber-scanned illumination and resonant-scanning detection. Furthermore, the pattern of non-resonant scanning of the ablation process or other laser therapy can be monitored by resonant scanning to insure that the desired pattern of illumination is followed.

An exemplary desirable approach for monitoring the laser ablation process and the depth of a fluorescent marker of disease in the tissue is multi-photon fluorescence imaging using relatively lower-power laser illumination, at ultra-short NIR optical frequencies. Advantages of using a non-confocal design for two-photon scanning fiber endoscopy are discussed below. The NIR light penetrates tissue with less optical loss of scattering and absorption than occurs when using ultraviolet or visible wavelengths. Thus, the NIR light creates a sharp focal point within tissue, with minimal losses even though the NIR wavelength is longer than that of either ultraviolet or visible light. Only at the focal point is there sufficient optical power to produce measurable two-photon absorption. Therefore, all fluorescent photons transmitted from the tissue can be additive to the signal for measuring the fluorescence. By scanning the tissue in two dimensions, a 2-D fluorescence image can be generated. Typically, the original 2-D image is repeated for different axial depths (slices) to generate a 3-D fluorescence image. Since the technique for two-photon fluorescence imaging is most often used to generate 3-D images, the fluorescence signal from below the surface of the tissue has a very high probability of scattering before emerging from the tissue surface. Because the scattering redirects the effective source point from the fluorescing specie within the illumination volume to the last point of scattering before emerging from the tissue, a confocal optical design for collecting the fluorescence signal can eliminate most of the signal. Only a small percentage of fluorescence photons that do not scatter from the illumination volume can be detected in a confocal optical design. Due to the fact that in non-confocal designs, the area for optical detection can be orders of magnitude larger, the fluorescence signal collection efficiencies can also be orders of magnitude greater for pseudo- and non-confocal optical designs compared to confocal designs. The greater signal strength that is achieved without increasing the illumination power is a significant advantage of a non-confocal design SFE probe for two-photon imaging of fluorescence from points below the surface of tissue.

The SFE probe will need to be moved into position within a patient's body before performing two-photon imaging. One method for accomplishing this is to guide the probe into position using an image created from backscatter of the illumination light from the surface of the tissue. To provide imaging illumination, it will be necessary to add white light that includes red, green, and blue components to the NIR light that is used for the two-photon excitation of visible fluorescence. By viewing an image formed with the visible backscattered light, an operator can determine the specific location of the SFE probe and determine where the two-photon imaging should be implemented. In this case, the two-photon SFE probe system would have a minimum of two imaging modes. The two-photon imaging mode would be using a single visible channel matched to the fluorescence emission, while the visible light imaging mode would use the red, green, and blue light sources and corresponding visible light detector(s). Moreover, it should be apparent that visual imaging can be either full-color using three laser or other red, green, and blue light sources such as light emitting diodes or filtered arc lamps, or monochrome using a single laser or other light source that emits a single color of visible light. Further, this probe system should be capable of switching between the two-photon and standard visual imaging on-demand from the operator, on a frame-by-frame basis, with a separate display for each image or the part of a single image that is gathered via two-photon imaging, linked to another part that is gathered as a standard visual image. Also one of the red, green, or blue visible light detectors can be used for two-photon fluorescence detection, or a separate optical detector can be used to generate the fluorescence images in response to two-photon light. It is also contemplated that this type of SFE probe would have the ability to overlay the two-photon fluorescence signal with a standard visible endoscopic image signal on a visual display.

FIG. 3 illustrates a two-photon SFE probe 60 that includes two or more illumination optical fibers 62 having proximal ends connected to a relatively high intensity NIR laser light source. Optionally, disposed on the distal ends of illumination optical fibers 62 are rod lenses 64, which may, for example, comprise GRIN lenses that compensate for any large chromatic shifts between illumination light and collected optical signal. Note that in this configuration, the rod lenses cooperate with lenses 70 and 72 to focus NIR light 74 in an illumination field 76. By rotating the probe, annular illumination field 76 can be created by the movement of the individual spots of illumination on the internal site. Objective lenses 70 and 72 also focus the two-photon light that is received from the tissue at the internal site onto the distal end of scanning optical fiber 66. The scanned region within illumination field 76 emits two-photon light 78 that enters scanning optical fibers 66, which is driven to scan in a desired scan pattern, at a resonant or near-resonant frequency of the scanning optical fiber, by piezoelectric two-axis driver 68. The two-photon light is conveyed back to an appropriate light sensor and filtered to remove the excitation light (not shown) disposed at the proximal end of the scanning optical fiber. Additional light may be collected by using a dual-clad optical fiber for both illumination and collection or simply adding additional collection optical fibers that surround the central resonant scanner. Ideally, illuminated field 76 is disposed very near or in contact with the distal end of SFE probe 60, and can be held or moved, for example, by suction or mechanical means (not shown). Contact via water-immersion or tissue-contact (oil-immersion) of the objective lens front surface may provide the image stability that may be required for high resolution imaging and high-power optics employed for administering laser therapies.

An exemplary application for SFE probe 60 would be in carrying out the technique discussed in an article entitled, “All-Optical Histology Using Ultrashort Laser Pulses,” by Philbert S. Tsai et al., Neuron, Vol. 39, 27-41, Jul. 3, 2003. The article explains a technique that was used to automate the three-dimensional histological analysis of brain tissue, demonstrating the use of femtosecond laser pulses to iteratively cut and image fixed, as well as fresh, brain tissue. Probe 60 and the methods disclosed herein can clearly be effectively employed in carrying out such work, and for many other applications.

### Exemplary Scanning Patterns

FIGS. 4A-4E illustrate several different scanning patterns that can be implemented by the scanning devices discussed herein, in connection with the various exemplary embodiments of the SFE probes. FIG. 4A illustrates a spiral scan pattern 90 that is achieved, for example, by driving the optical scanning fiber with a triangle modulated sine wave along a first axis, and with a triangle modulated cosine wave along an orthogonal second axis. FIG. 4B illustrates a linear scanning pattern 92 that rotates about a central axis; FIG. 4C illustrates an exemplary raster scan pattern 94; FIG. 4D illustrates an exemplary propeller scan pattern 96 that rotates about a central axis; and FIG. 4E illustrates an exemplary Lissajous scan pattern 98. Many other scanning patterns can be achieved by driving the actuator for the scanning device with one or more appropriate drive signals.

### Exemplary Embodiment with Additional Optical Fibers

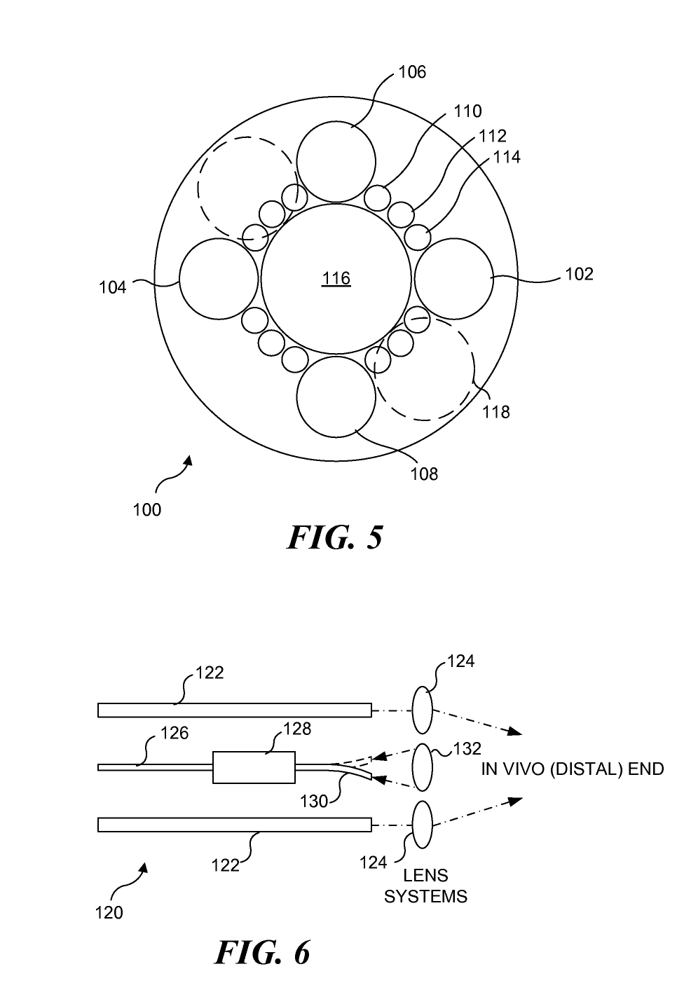

The exemplary embodiments for SFE probes discussed above have all included one or more illumination optical fibers for conveying light to the tissue at the internal site being scanned. FIG. 5 illustrates a distal end of an exemplary SFE probe 100 that includes four illumination optical fibers 102, 104, 106, and 108 disposed peripherally around a central region 116, where a scanning device employed for imaging and/or optical diagnoses is disposed. In addition, SFE probe 100 includes collection fibers 110, 112, and 114 disposed between adjacent pairs of the optical illumination fibers. These collection fibers are stationary and are operative to collect red, green, and blue light or other wavelength light received from the tissue at an internal site. Alternatively, the collection fibers can be replaced with optical detectors or sensors that are disposed proximate to the distal end of the SFE probe. The light collected by the collection fibers (or by the alternative optical detectors or sensors) can be employed for imaging or determining a condition of the tissue at the internal site as part of the task of performing diagnosis or monitoring the tissue during or after optical therapy is applied via the illumination optical fibers. Alternatively, disposed between these optical illumination fibers, larger lumens or conduits 118 can be used for introducing fluids to and removing fluids from the internal site, by varying an applied pressure, for such purposes as cleaning, rinsing, optical labeling, staining tissue, or removing cells or secretions. The lumens or conduits may be of sufficient size to permit the passage of biopsy tools, needles, or brushes.

As shown in an exemplary embodiment of an SFE probe 120 in FIG. 6, illumination optical fibers 122 can each be provided with separate lenses 124, while a lens 132 focuses light from the internal site into the distal end of a scanning optical fiber 130 that is resonantly driven by an actuator 128. Light entering the scanning optical fiber is then conveyed proximally to an appropriate detector or sensor (not shown) through an optical fiber 126.

### Exemplary System for Determining a Distance Between Scanner and Tissue Surface

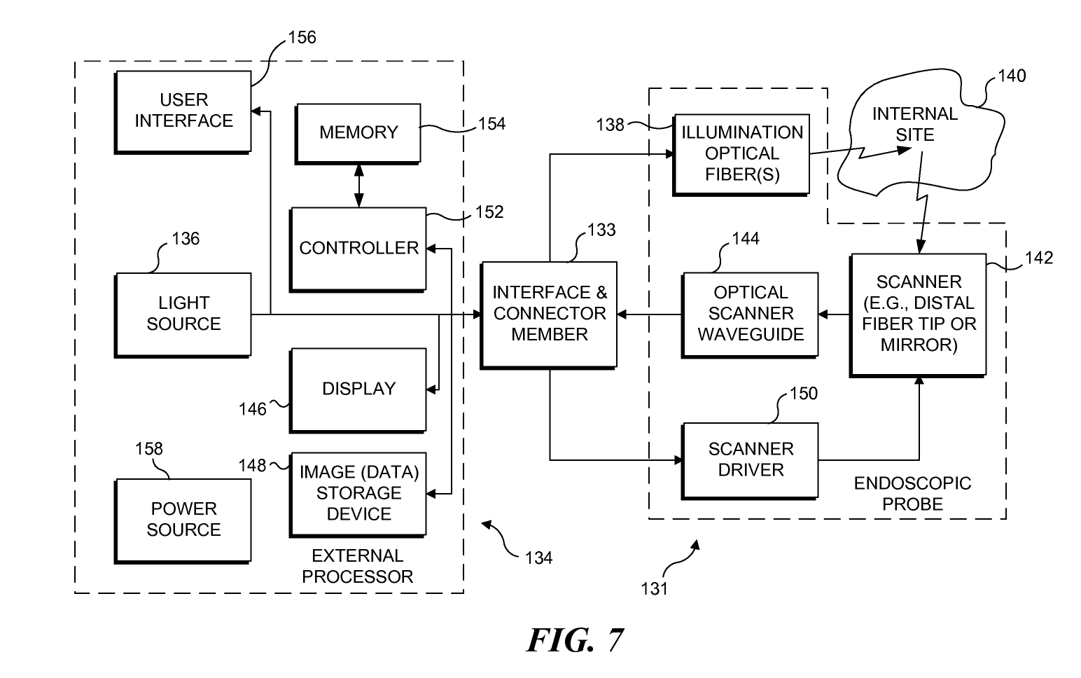

FIG. 7 is a simplified block diagram illustrating an exemplary external control system 134 that is coupled to an SFE probe 131 through an interface and connector member 133. SFE probe 131 and external control system 134 are generally configured as described below to determine a distance between the probe and tissue at an internal site 140, as a function of specular reflections from the surface of the tissue. SFE probe 131 includes one or more illumination optical fibers 138 that convey illumination light to internal site 140, generally as described above. The illumination light that is reflected from the surface of internal site 140 has two primary components, including a diffuse reflected portion and a spectral reflected portion. The diffuse reflected portion spreads over a wider angle relative to the direction at which the illumination light is incident on the surface of the tissue at the internal site, and this angle is commonly referred to as the Lambertian angle. In contrast, the specular reflected portion is reflected from the tissue surface at the same angle at which it was incident to the surface. The percentage of light that is absorbed by the tissue, or is diffusely reflected, or is specularly reflected, depends on the material properties of the tissue surface and on the wavelength of the incident illumination light. Shiny or wet tissue surfaces generally have a higher spectral reflection component than do matte surfaces.

The light reflected from the tissue surface at internal site 140 enters a scanner 142, which, depending upon the exemplary embodiment employed, can be the distal end of a scanning optical fiber or a scanning mirror, as discussed above. This light that is thus collected by the scanner is conveyed through an optical scanner waveguide 144, e.g., an optical fiber, to interface and connector member 133, which couples the light into external control system 134. Scanner 142 is driven by a scanner driver or actuator 150 in response to a signal received from the external control system via interface and connector member 133.

External control system 134 includes a light source 136, which will typically comprise one or more laser sources that produce coherent light at one or more wavelengths for input to the proximal ends of illumination optical fibers 138, conveyed through interface and connector member 133. Optionally, light source 136 may also include any one or more of red, green, and blue light sources, an IR light source, and an ultraviolet light source. The light source can be switchable from a continuous mode to a pulse mode, depending upon the application, or depending upon when the light sources are used, e.g., for imaging in one frame, rendering therapy in another frame, and for still other purposes in yet another frame of a sequence of frames. If light of a plurality of different wavelengths is produced by light source 136, a combiner may be employed to combine the different wavelengths, or the light of different wavelengths may be conveyed separately through different illumination optical fibers 138.

External control system also includes a controller 152, which may, for example, comprise one or more microprocessors, an application specific integrated circuit (ASIC), a gate array, a logic device, or other form of computing device. Controller 152 is employed to control scanner driver 150 by providing one or more appropriate drive signal(s) to achieve a desired scan pattern by driving scanner 142 at a resonant or near-resonant frequency. Controller 152 may be coupled to a memory 154 in which machine language instructions are stored for controlling a central processing unit (CPU) or other computing device comprising controller 152 to carry out the functionality of the external control system, as disclosed herein. A display 146 is typically included to enable images of internal site 140 produced in response to the light received from the internal site by the scanner to be viewed by an operator. Optionally, an image storage device 148 can be provided for storing data corresponding to the image of the internal site derived from the light conveyed from scanner 142 for subsequent additional processing, display, or archival purposes. A user interface 156 is provided to enable a user to enter control parameters, or carry out various desired functions with SFE probe 131, and for controlling the SFE probe as desired. The user interface can include a keyboard, a keypad, a pointing device, or other appropriate mechanism for input of user control actions, selections, and values. A power source 158 provides appropriate voltage and current levels to energize each of the electronic components included within external control 134. It will be understood by those of ordinary skill in the art that this exemplary embodiment for external control system 134 is not intended to be limiting, since many other components and configurations usable for coupling to and controlling an SFE probe like those disclosed herein can be employed with equal facility.

Further functional details of external control system 134 are shown in FIG. 9. To enable both imaging and distance measurement using external control system 134, controller 152 includes a spectral light/dispersed light separator 166 that is used for separating the spectral portion of the light received by the scanner from the diffuse portion. In response to the diffuse portion of the light, a signal is produced and input to an image processor 162. Signals produced in response to the spectral portion of the light are conveyed to a surface geometry processor 164. Image data from image processor 162 are output from controller 152 and employed to drive display 146 to enable an operator to view images of the internal site. However, surface geometry processor 164 can also transmit data to display 146, and part of this data can be dedicated to a geometrical data output 168. Alternatively, geometry information can be superimposed on the conventional image of the internal site provided on display 146, or may be separated and shown on a different display, or at a different time than the image of the internal site.

It should be understood that although image processor 162 can primarily use the dispersed portion of light reflected from the surface of the tissue at the internal site, the image processor can also use the spectral portion of the light. Further, surface geometry processor 164 can make some use of the dispersed light signals from spectral light/dispersed light separator 166. Furthermore, spectral light/dispersed light separator 166, image processor 162, and service geometry processor 164 can comprise modules that include hardware and/or software. Machine language programming to carry out the functions such as the measurement of spectral light can be provided to controller 152 from a storage medium 170. Storage medium 170 can comprise, for example, an optical disk, such as a compact disc-read only memory (CD-ROM), or a digital versatile disk (DVD) that is read by a media drive 172. Other alternatives for input of these program instructions and/or data include magnetic recording media, e.g., a floppy disk, or a removable hard disk, as well as a remote data source coupled to the controller via a network, a wireless connection, or the Internet.

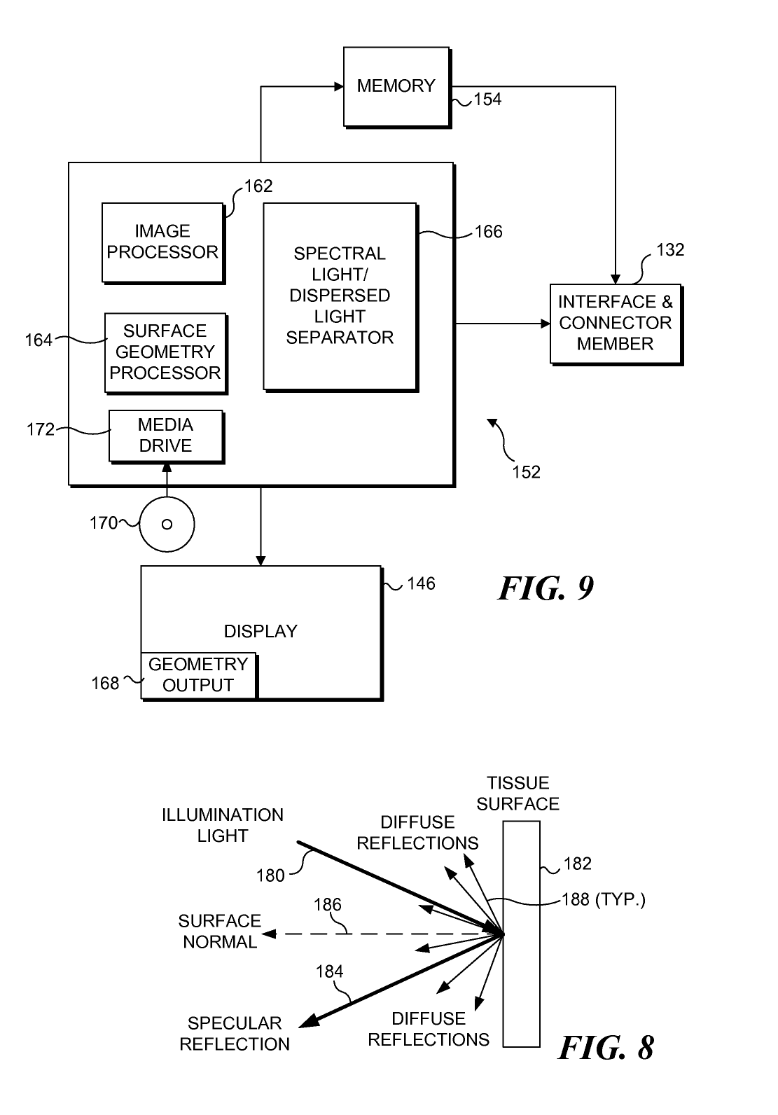

A schematic representation illustrating the relationship between illumination light 180 incident on a tissue surface 182, a specular reflection 184, and diffuse reflections 188, is illustrated in FIG. 8. The angle of specular reflection 184 relative to a surface normal 186 is equal to the angle between illumination light 180 and the surface normal. In contrast, diffuse reflections 188 are distributed at various other angles relative to the surface normal. Some of illumination light 180 that strikes tissue surface 182 is absorbed by the tissue and not reflected.

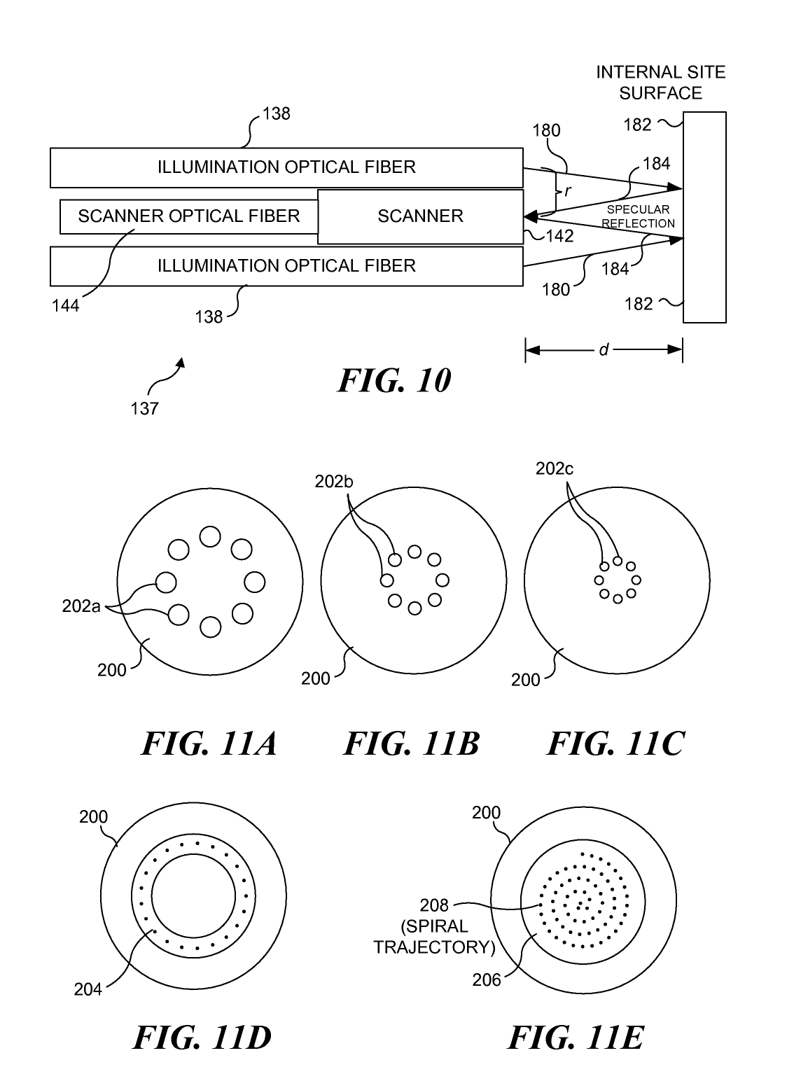

An important reason to measure specular reflection from tissue surface 182 is that the distance, d, between the distal end of the SFE probe and the tissue surface can readily be determined as a function of the specular reflection. FIG. 10 schematically illustrates the geometrical relationship between the distal end of SFE probe 137 and tissue surface 182 in connection with illumination light 180 that is emitted from illumination optical fibers 138. As shown in this Figure, specular light 184 enters scanner 142 and is conveyed proximally through scanner optical fiber 144. In FIGS. 7 and 9, the distance d is really determined by controller 152 as a function of the scanner position relative to illumination light 180. In one position of the scanner, primarily diffuse light will be received by the scanner, while when the scanner is at a different second position, primarily specular light will be received and input to the scanner. Since the specular portion of the light that is input to the scanner may have a substantially higher energy than the diffuse portion, the signal response of the scanner will be significantly greater when receiving the specular light than when receiving diffuse light from the internal site. Spectral light/dispersed light separator 166 in controller 152 (FIG. 9) can include a threshold for separating the diffuse portion from the spectral portion. Known image processing techniques can readily be used to identify a perimeter of the spectral portion of the light, an area, a cross-sectional size, a centroid, and the like.

FIG. 11A illustrates an image 200 that is produced when SFE probe 137 is employed to image a tissue surface that is normal to the axis of the SFE probe. In the image shown in FIG. 11A, eight bright spots 202a define a spectral reflection pattern at the center of the image if the illumination optical fibers are symmetrically positioned about the scanning center of the scanner. The specular reflection pattern can be problematic during image formation using SFE probe 137, because the high intensity of the spectral reflection relative to the diffuse reflected portion (on which the image is primarily based) tends to wash out or otherwise distort the desired image formation. Separation of the spectral portion, identification of the spectral pattern, filtering, and/or other image processing steps can be taken to reduce the effects of spectral reflection in the displayed image of the tissue surface.

The size of the spectral reflection pattern is determined at least in part by the magnitude of the scan provided by SFE probe 137 during imaging, which can be expressed as angular field of view (FOV), and by the distance between the scanner and tissue surface 182. When scanning with a constant FOV, the spectral reflection pattern will be larger when the scanner is closer to the surface and smaller as it is moved away from the surface, as will be evident by comparing the spectral reflection patterns comprising spots 202b and 202c in images 200 of FIGS. 11B and 11C, respectively, with the spectral reflection pattern in FIG. 11A. Thus, the spectral reflection pattern size can be used to determine the distance between the distal end of the SFE probe and the tissue surface.

In the simple case discussed above, the tissue surface is assumed to be normal to the scan direction. Certain parameters for SFE probe 137 are known. For example, r is the known distance between the center of the scanning optical fiber and illumination optical fibers 138; θₘₐₓ is the known maximum field of view of the scanner; and Sₘₐₓ is the known number of scan spirals that form an image (assuming that a spiral scan pattern is used). Using these known parameters, the distance d between the distal end of the probe and the tissue surface can be calculated by capturing the image and performing a binary threshold on the image data so that pixels in the image that receive specular reflections are assigned a binary value 0. At least one connected image object corresponding to the distal end of the scanner should be identified in the connected image. A pixel at the center of this connected object or to which it is closest is then determined, and remapping is used to determine a scan angle between the center of the image scan and the center or centroid of the connected object, S_{c}. The scan angle can be saved in a remapping lookup table for each pixel.

The distance d to the surface of the tissue from the distal end of the probe is computed as:

d=r/(2*tan(θ_{c})) (1)

Where the image includes more than one connected object, which occurs because a plurality of illumination optical fibers are employed, the distance from the distal end of the SFE probe to the tissue surface can be computed for each connected object and averaged to give a more accurate value. The calculation becomes slightly more complex if the normal to the tissue surface is not aligned with the longitudinal axis of the SFE probe. However, use of a plurality of illumination optical fibers readily enables the distance to be calculated relative to the central scanner.

An additional reason to measure the specular light reflection from tissue surface 182 is that at a fixed distance, d, the pattern of illumination can be monitored as that pattern is modified by non-resonant scanning. As shown in image 200 of FIG. 11D, when the illumination beams are rotated with respect to the tissue orientation, the individual spots of FIGS. 11A-11C will create an annulus of illumination 204. A corresponding illumination field 76 is also illustrated in FIG. 3 when the entire SFE probe 60 is rotated with respect to the internal site. As shown in image 200 of FIG. 11E, when the illumination beams are directed inward toward the central optical axis concurrently with rotation with respect to the tissue, the individual spots of FIGS. 11A-11C will merge and create an area-filling pattern of illumination 206, following a spiral trajectory 208 over time. The purpose of monitoring the varying non-resonant scan patterns of illumination is to provide therapeutic illumination over the entire area of interest with knowledge that no gaps of illumination have occurred.

### Exemplary SFE Probe with Fixed Illumination Optical Fibers

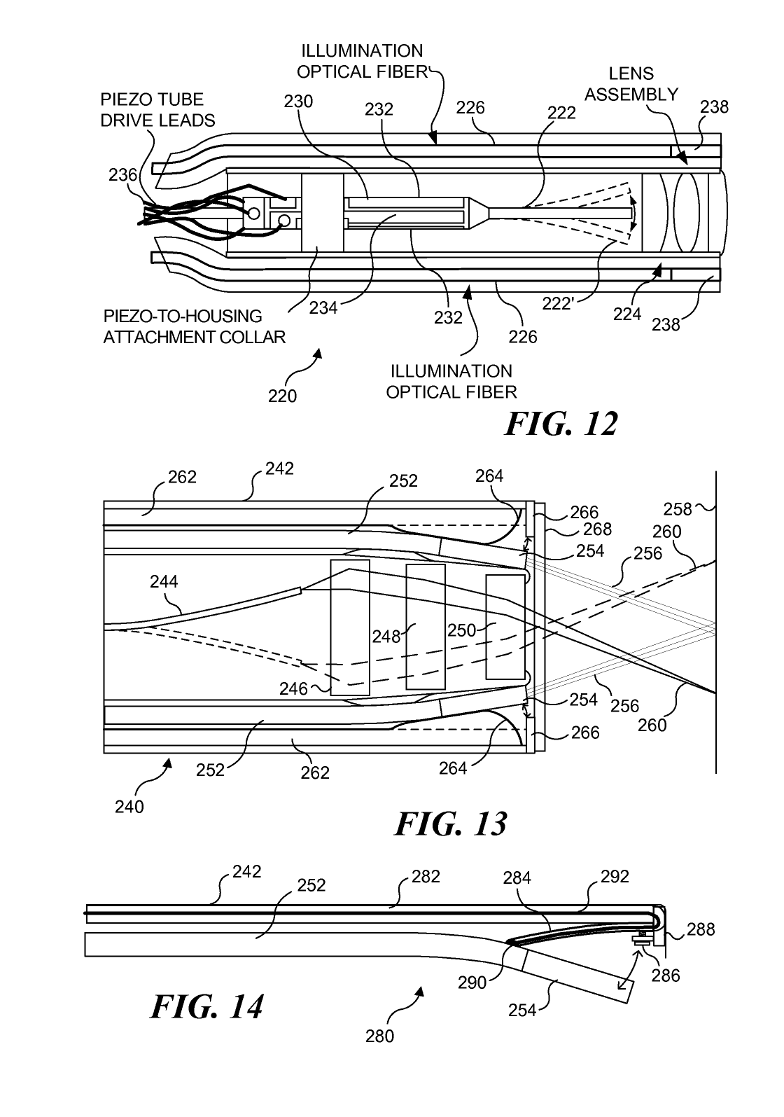

FIG. 12 illustrates an exemplary SFE probe 220 that includes a scanning optical fiber 222, which is driven to scan in a desired scan pattern at or near its resonant frequency, as indicated by its position in phantom view at reference numeral 222′. A lens assembly 224 is provided at the distal end of SFE probe 220 and is employed for focusing the light being received by scanning optical fiber 222. Two or more illumination optical fibers 226 are disposed peripherally around scanning optical fiber 222 and are used for conveying illumination light or therapy light to a desired internal site within the body of a patient (not shown). In this exemplary embodiment, illumination optical fibers 226 are fixed in position but may each include an optional rod lens 238 disposed at a distal end for more effectively focusing the light emitted from the illumination optical fiber. Scanning optical fiber 222 is driven in a desired pattern by a piezoelectric tube actuator 230 relative to two orthogonal axes, in response to drive signals supplied to electrodes 232 and 234 through electrical leads 236, which extend proximally from SFE probe 220. A single-axis (linear) scan pattern can, for example, be generated by applying voltage to one or to opposing electrodes 232 of piezoceramic tube actuator 230. By applying an oscillating periodic voltage (e.g., a sine wave) having a frequency at or near the mechanical resonant frequency of the base-excited scanning optical fiber cantilever to the actuator through electrical leads 236, the amplitude of the tip motion can be mechanically amplified due to the mechanical resonance of the scanning optical fiber cantilever. Furthermore, for example, the concurrent application of a second periodic voltage (a cosine wave) to electrodes 234 (which are orthogonal to electrodes 232) on the actuator, at the same or slightly different resonant frequency, causes the resonating fiber tip to move in an elliptical scanning pattern.

A signal useful for producing an image is generated by the optical fiber scanner shown in FIG. 12 by directing the light from illumination optical fibers 226 onto a region at the internal site. Light received from the region by the scanning optical fiber cantilever is focused using imaging lenses 224. Typically, the imaging lenses focus and magnify a scanned portion of the internal site, by resonantly scanning the site with either a linear (one-dimensional), or with spiral or elliptical (two-dimensional) patterns. By varying the amplitude of the voltages applied to the actuator during the elliptical scan, a two-dimensional (2-D) space-filling scanning pattern is formed. Illumination optical fibers 226 that surround the optical fiber scanner are used to provide the illumination light that is reflected from the tissue at the internal site for generating the 2-D image or for collecting light used to evaluate other parameters of the tissue. Typically, illumination optical fibers 226 are large-core multimode optical fibers having a high-numerical aperture (NA), which provides excellent light delivering efficiency. Alternatively, new large-core, but singlemode photonic crystal optical fibers may be used to deliver 0.5 Watt or more of optical power. In contrast, optical fiber cantilever 222, which is used for scanned light collection or for imaging, can comprise, for example, a small diameter singlemode core as part of a multi-clad optical fiber that has a large NA multimode inner cladding, for added functionality.

In many applications, the use of fixed illumination optical fibers will not be of any disadvantage, since there will be no need to modify the direction or focal point of the illumination optical fibers as they emit light directed toward the internal site. However, in other applications, it may be necessary to selectively modify the direction in which light is emitted from the illumination optical fibers, for example, by changing the angular orientation of the illumination optical fibers, and in some cases, modifying the focal point of the illumination optical fibers.

For example, in one exemplary application of an SFE probe, optical properties of tissue can be measured by illuminating the tissue surface with a point source or a focused beam of light and then measuring the reflectance of the tissue as a function of wavelength and spatial distance from the point source. By measuring the total and relative spatial distribution of steady-state diffuse reflectance from the tissue surface, the local tissue optical properties of transport scattering coefficient and absorption coefficients can be calculated. Diffusion theory of light transport in tissue is the theoretical framework for modeling light-tissue interactions in the optical window of red to IR optical frequencies or in the 600 nm to 1300 nm wavelength range. The optical properties of absorption and scattering coefficients within this optical window are useful for determining how much laser light is reaching different depths in the tissue and what fraction of this light is being absorbed, for laser therapies such as photodynamic therapy, laser heating, and laser ablation. By illuminating the tissue with a plurality of different wavelengths of light within this optical window and using circular or spiral scanning patterns or other scanning patterns of known radial extent, it is possible to more accurately measure the penetration depth and spatial distribution of the optical irradiation within the tissue, calculate the absorption or concentration of light absorbers within the tissue, and monitor therapeutic changes in tissue properties with minimal invasiveness to the surrounding tissue.

A point of illumination can be delivered using one or more optical fibers, while the spatial distribution of the diffuse reflectance can be detected using either a scanned optical fiber, as shown in FIG. 1, or a mirror scan system, as shown in FIG. 2. Optical lens systems can be used between the illumination optical fiber and the tissue surface to focus the irradiating light to illuminate a smaller area on the tissue surface, as shown in FIGS. 3 and 6. The illuminating light from each optical fiber can be focused on the tissue surface as individual point sources or the light can be combined into a single central point of illumination, as shown in FIG. 8. Ideally, if a single and central point of light illumination is used, then it may be desirable to employ scanned detection in a slowly expanding spiral or simply using a circular pattern at the desired radial distance from this central illumination point source, using a resonant fiber scanner or mirror scanner like those discussed above. Because the each scan cycle in a circular pattern is at a constant radial distance from the central point source, all light detected from this circular pattern can be averaged (to reduce noise in the measurement), producing an accurate determination of diffuse reflectance at this specific radial distance R(ρ). The radial distance (ρ) can be varied over a wide range by simply changing the scan angle of the resonant scanner. By measuring the optical power within the optical fiber used for illumination, the relative spatial distribution of light reflectance can be measured for a wide range of radial distances. Finally, an absolute measure of reflectance from the single point source can be estimated by integrating these spatial measurements over this limited range of radii that were measured and then scaling this value to determine an estimated reflectance profile if all radii were to be measured, assuming homogenous and semi-infinite tissue properties.

The relationship between measured optical properties of tissue and these measurements of absolute and relative spatial distribution of tissue surface reflectance from a point source provides excellent correlation to in vitro measurements, even when simplifying assumptions of the tissue are being made when applying diffusion theory. A person of ordinary skill in the art will readily understand how these measurements can be carried out and how to apply these optical properties of tissue and further details need not be provided. By way of demonstrating the knowledge of those of skill in this art, these mathematical and empirical relationships and the measurement techniques are discussed in an article entitled, “Quantitative reflectance spectrophotometry for the noninvasive measurement of photosensitizer concentration in tissue during photodynamic therapy,” by M. S. Patterson, E. Schwartz, and B. C. Wilson in Photodynamic Therapy: Mechanisms, Proc. SPIE vol. 1065, pp. 115-122 (1989); and in an article detailing an extension of this work, by B. C. Wilson and S. L. Jacques entitled “Optical reflectance and transmittance of tissues: principles and applications,” IEEE Journal of Quantum Electronics, vol. 26, no. 12, pp. 2186-2199 (December 1990). However, the conventional implementation of measuring tissue optical properties disclosed in the two references have limitations when applied in medical practice. A major challenge for minimally-invasive medical instrumentation is the required small size and highly accurate optical measurements. Even the authors of the technique disclosed in these references have identified two limitations of their approach in practice, as noted in an invited article entitled, “Applications of time-resolved light scattering measurements to photodynamic therapy dosimetry,” by M. S. Patterson, J. D. Moulton, B. C. Wilson, and B. Chance, in Photodynamic Therapy: Mechanisms II, Proc. SPIE vol. 1203, pp. 62-75 (1990). The stated two limitations of the technique are that the diffuse reflectance measurements must be made at a number of different locations at the tissue surface, and that these measurements must be quantitatively related to the incident irradiation. The approach disclosed in these references used an integrating sphere to measure R and a traveling stage for measuring R(ρ), while a camera detector array is only proposed for a future embodiment. However, using the present novel approach disclosed herein, it is contemplated that R(ρ) can be measured using a resonant scan system after illumination by one or more optical fibers. Each optical fiber can be metered to quantitatively relate this incident illumination to the measured R(ρ). A specific exemplary embodiment uses the illumination at a single and central point source, with R(ρ) detection employing a circular or spiral scan pattern that averages the detected diffuse reflectance R at a slowly varying distance (ρ) from the central point source. The resonant scan detection system can provide a wide and variable range of measured R(ρ) by simply adjusting the drive signal to the scanner to make the scan grow larger or smaller. For the same small size of less than 2 mm in diameter, the resonant fiber scanner has been demonstrated to provide more than twice the image resolution of standard coherent fiber bundle or a micro-camera chip for flexible endoscopy, as reported by E. J. Seibel, R. S. Johnston, and C. D. Melville in an article entitled, “A full color scanning fiber endoscope,” Optical Fibers and Sensors for Medical Diagnostics and Treatment Applications VI, Proc. SPIE vol. 6083, ID#608303 (2006). Therefore, the R(ρ) measurement can be made at a number of locations on the tissue surface deep within the human body using a small and accurate resonant fiber scanning device that overcomes technical challenges that previously have made the local measurement of optical properties impractical.

### Exemplary Embodiments Having Non-Resonantly Movable Illumination Optical Fibers

FIG. 13 illustrates the distal end of an SFE probe 240 that includes a first exemplary embodiment that enables illumination optical fibers 252 to be selectively moved (i.e., slowly and not at a resonant rate), so that the direction along which illumination light 256 is emitted from optional rod lenses 254 can be varied as desired. SFE probe 240 has a housing 242 in which a scanning optical fiber 244 is driven to move at a resonant or near-resonant frequency and in a desired scanning pattern by a driver (not shown in this view). Light 260 from an internal site 258 is received by scanning optical fiber 244 after the light has passed through a transparent cover 268 disposed at the distal end of the SFE probe, and then through lenses 250, 248, and 246. The distal end of illumination optical fibers 252 (including rod lens 254—if provided) is selectively moved radially inward relative to the longitudinal center of SFE probe 240 by inflating a balloon 264 with a fluid conveyed through one or more lumens 262 that extend from a pressurized source disposed adjacent to the proximal end of the SFE probe. The pressurized fluid within balloon 264 inflates it so that it applies a force against the distal end of illumination optical fiber 252 directed radially inward.

The natural stiffness of the illumination optical fibers tends to resist the deflecting force applied by balloon 264 as it is inflated. Alternatively, a spring (e.g., a helical spring, or flat spring running along the longitudinal axis of the illumination optical fiber) or other mechanism that exerts an outwardly directed force can be provided to bias the distal ends of the illumination optical fibers radially outwardly, so as to resist the force applied by the balloon. As the balloon is deflated, the distal ends of the illumination optical fibers are moved radially outwardly by this biasing force. A ring (or one or more tabs) 266 can serve as stops that preclude the radially outward movement of the illumination optical fibers/rod lenses beyond a desired limit.

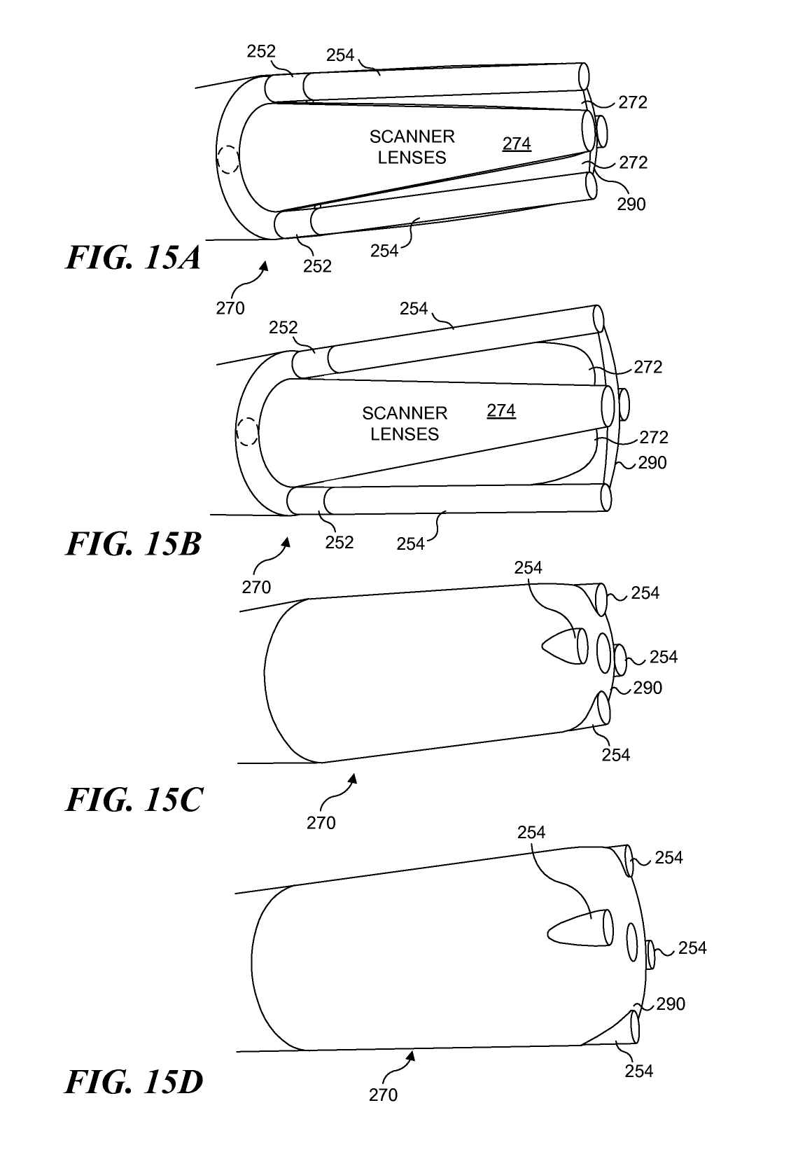

FIG. 15A is a cut-away view of the distal end of an alternative SFE probe 270 showing illumination optical fibers 252 and rod lenses 254 disposed and angled radially inward at a position wherein a balloon 272 (disposed between the illumination optical fibers and the outer surface of a scanner lens housing 274) is generally deflated. As shown in FIG. 15B, the illumination optical fiber and rod lenses are angled more radially outwardly in response to balloon 272 being selectively inflated with a pressurized fluid supplied from a source (not shown) disposed at the proximal end of the SFE probe. FIGS. 15C and 15D are isometric views of SFE probe 270 respectively corresponding to FIGS. 15A and 15B, showing an elastomeric cover 276.

FIG. 14 illustrates a partial cut-away view of an exemplary SFE probe 280, which includes one or more illumination optical fibers 252 having an optional rod lens 254. However, SFE probe 280 does not include a balloon for moving the one or more illumination optical fibers to a different angular orientation. Instead, it employs an electromechanical actuator or piezoelectric bimorph bender 284 that is held in place by a clamping screw/support 286 that extends proximally of a stop 288. Stop 288 prevents the radially outward motion of the illumination optical fiber(s) beyond a desired limit. An electric lead 292 extends through a lumen 282 from a power source (not shown) that is disposed at a proximal end of the SFE probe. The current in electrical lead 288 is extremely low and the voltage is relatively low; and one or both of these parameters can be selectively controlled to move the illumination optical fiber(s) with the electromechanical actuator or piezoelectric bimorph bender 284 to a desired angular orientation. A low-friction and non-conductive tip 290 is provided at the end of the electromechanical actuator or piezoelectric bimorph bender where it contacts an illumination optical fiber. A spring (not shown) longitudinally extending along the illumination optical fiber, or a helical spring (also not shown) extending radially and connected to housing 242 can optionally be employed to provide a radially outwardly directed biasing force opposing the radially inwardly directed force provided by electromechanical actuator or piezoelectric bimorph bender 284.

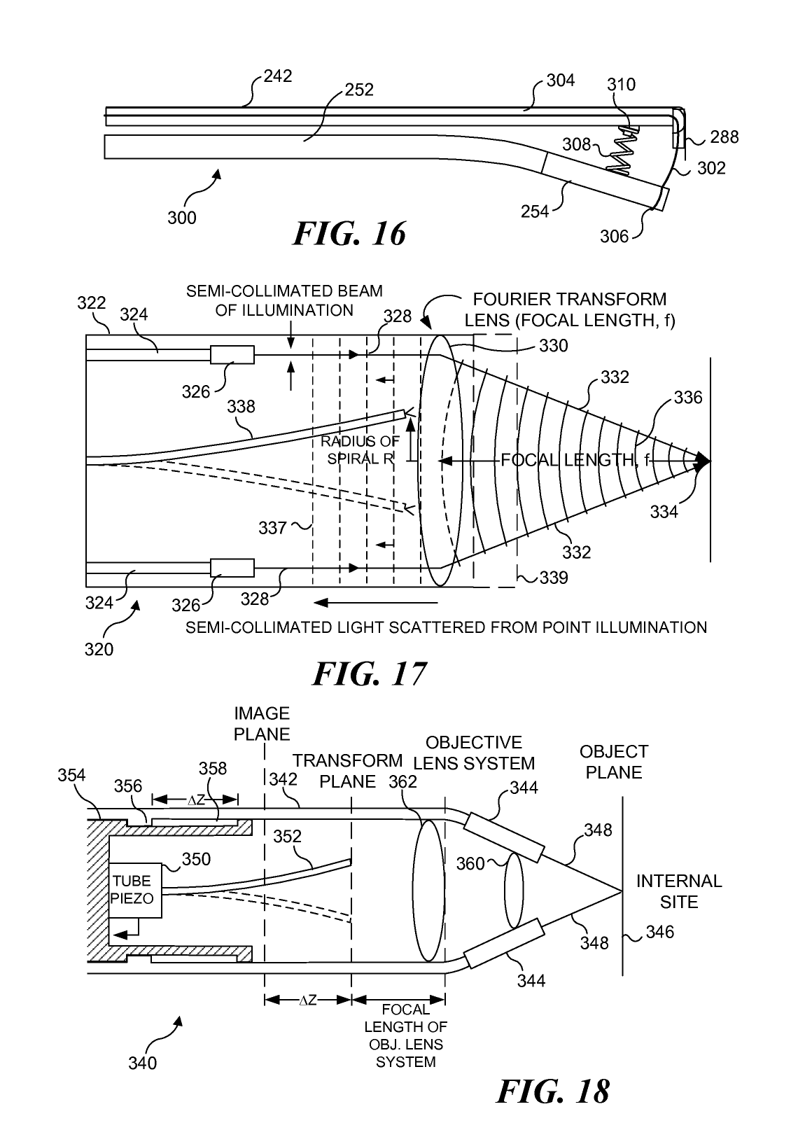

A partial cut-away view of the distal end of still another exemplary embodiment of an SFE probe 300 having means for moving the illumination optical fibers is illustrated in FIG. 16. Specifically, SFE probe 300 includes a cable or wire 302 that is coupled around illumination optical fiber 252 (or around rod lens 254) via a loop 306 and which extends proximally through a lumen 304. A stop 288 prevents the radially outward travel of the illumination optical fiber or rod lens beyond a desired limit. A helical spring 308 is coupled to a nib 310 on the inside surface of housing 242 and is used for producing a biasing force directed radially inwardly against illumination optical fiber 252 (or rod lens 254). As cable or wire 302 is pulled, the force applied to the illumination optical fiber or rod lens compresses helical spring 308 and moves the illumination optical fiber radially outwardly to change its angular orientation and the direction in which light emitted from the illumination optical fiber travels toward an internal site. When the cable or wire tension is eased, the helical spring moves the illumination optical fiber 252 and rod lens 254 radially inward.

### Exemplary Embodiment for Capturing Different Scattering Angles

The distal end of an exemplary SFE probe 320 designed to endoscopically measure scattering angles at a Fourier transform plane using one or more illumination optical fibers 324 is illustrated in FIG. 17. The distal end of each illumination optical fiber is provided with a rod lens 326 to provide an effective point source illumination. Also included within the SFE probe is a resonant scanning optical fiber 338. SFE probe 320 is disposed in a housing 322 that supports a Fourier transform lens 330 having a focal length, f. Light 328 conveyed through the illumination optical fibers can be collimated or semi-collimated by rod lenses 326 and is directed by Fourier transform lens 330 as light 332, to a localized region 334 on the internal site. The rod lenses can be configured to adjust the location and degree of collimation of the illumination light effective point source. Light 336 that is scattered from particles at region 334 enters Fourier transform lens 330, which produces semi-collimated light 337 that is collected by scanning optical fiber 338. The signal produced by a detector (not shown in this Figure) that is coupled to the proximal end of the scanning optical fiber is used to determine the scattering angle of the collected light. This scattering angle is indicative of the relative size of the particles that caused the scattering.

Measurement of scattering angle can be very useful for diagnosing tissue condition at the internal site. For example, it is well known that cancer cells tend to have a substantially larger nuclear-to-cytoplasmic ratio (nuclear diameter divided by cell diameter) than normal cells. Accordingly, measuring the normalized scattering of light from the internal site enables a sharper peak to be identified for the larger cancer cell nuclei, compared to that of normal cell nuclei.

The technique that can be employed for determining scattering angle is well known to those of ordinary skill in this art and further details need not be provided in this disclosure. For example, an exemplary technique is disclosed in an article entitled, “Fourier-Domain Angle-Resolved Low Coherence Interferometry through an Endoscopic Fiber Bundle for Light-Scattering Spectroscopy,” by John W. Pyhtila, Jeffrey D. Boyer, Kevin J. Chalut, and Adam Wax, OPTICS LETTERS, Vol. 31, No. 6, Mar. 15, 2006, and in an article entitled, “Determining Nuclear Morphology Using an Improved Angle-Resolved Low Coherence Interferometry System,” by John W. Pyhtila, Robert N. Graf, and Adam Wax, OPTICS EXPRESS 3473, Vol. 11, No. 25, Dec. 15, 2003. The conventional approach disclosed in these two articles uses an optical fiber bundle and a bare stationary singlemode optical fiber disposed behind a ball-lens objective and does not have the ability to image with the SFE. However, the articles note the ability to select depth of light scattering within tissue using coherence effects, while it is contemplated that instead, standard polarization filtering could be used for this purpose. Furthermore, unlike the prior art technique disclosed in these articles, the present approach employs scanned light to measure and map tissue optical properties, which is more efficient.

An exemplary range of scattering angles for cell components such as the nuclei of cells that can be measured with the system shown in FIG. 17 is from about 5° to about 30°. The minimum scattering angle is limited by the focal length of the Fourier transform lens, which in this exemplary embodiment, is about 3 mm, while the maximum scattering angle is limited by the radius of the spiral scanning pattern, r, which is produced by scanning optical fiber 338, and is about 1 mm for a maximum scattering angle of 30°. To achieve this radius for the spiral scanning pattern, it will be necessary to increase the length of the scanning optical fiber compared to scanning optical fibers discussed above in other SFE probes (assuming that the diameter of the scanning optical fiber is maintained at a nominal value of about 125 μm) and/or increase the magnitude of the drive signal applied to the scanning optical fiber actuator in excess of 20 volts.

The scanning optical fiber will, in one exemplary embodiment, comprise a dual clad, singlemode optical fiber to ensure that the collection efficiency is increased for sampling scattering at various angles, while enabling high resolution imaging of the internal site. For example, in sequential frames, the scattering angle can be measured, and the internal site then imaged using the scanning optical fiber. Alternatively, a collimating micro-lens (e.g., a ball lens) can be provided at the distal tip of scanning optical fiber 338 so that the scanning optical fiber can more effectively be used for imaging the internal site, while one or more additional optical fibers (e.g., an optical fiber not being used to provide illumination light) can respond to the backscattered light to measure the scattering angle. Yet another alternative embodiment for both imaging the internal site and capturing different scattering angles for particles at the internal site is shown in FIG. 18, which is discussed below.

By using a plurality of illumination optical fibers, it is possible to employ illumination light at two or more substantially different wavelengths, to enable different penetration depths in tissue at the internal site and to change the relative scattering angle for tissue structures at the different depths. Also, using a plurality of illumination optical fibers 324 can facilitate providing illumination light having different optical polarizations and may reduce noise from laser or other light source speckle. To differentiate light scattering from the tissue surface regions (e.g., epithelial layer) and not from deeper regions of the tissue, polarization filtering can be used. To implement this technique of filtering out multiply-scattered photons from single-scattered photons or only a few-scattered photons, linearly polarized illumination light can be used. In this case illumination fibers 324 can be polarization-preserving optical fibers. A wire-grid polarization filter can optionally be placed at the distal tip of the probe 339 with characteristics that match the polarization of the illumination and will then attenuate any polarization shifts due to multiple scattering events.

With reference to FIG. 18, an exemplary embodiment of an SFE probe 340 is illustrated that is designed to both image an internal site 346 and capture the different scattering angles for particles within tissue at the internal site. In this embodiment, illumination optical fibers 342 convey light from one or more different light sources through rod lenses 344, which at least partially collimate the light and direct it at the internal site as light 348. A piezoelectric tube actuator 350 is mounted to a sliding collar 354 that includes an annular groove 358, which limits a longitudinal movement of the piezoelectric tube actuator and scanning optical fiber 352 to a total distance equal to Δz, which corresponds to the distance between the image plane and the transform plane. Illumination optical fibers 342 include a stop 356, which engages groove 358. Alternatively, a similar sliding arrangement could be employed to move the illuminating delivery system and objective lens system, which includes lenses 360 and 362, in one exemplary embodiment.

When collecting light scattered from the internal site to determine scattering angles, the distal end of scanning optical fiber 352 is positioned on the transform plane, while when imaging the internal site, the piezoelectric tube actuator and scanning optical fiber are moved longitudinally, so that the distal end of the scanning optical fiber is disposed on the image plane. A similar movement over a distance Δz could instead be applied to the objective lens system to position it so as to change the distal end of the scanning optical fiber to be at either the transform plane or the image plane, depending upon whether a scattering angle is being determined, or the object plane at the internal site is being imaged.

### Exemplary Embodiment for Measuring/Mapping Local Diffuse Reflectance

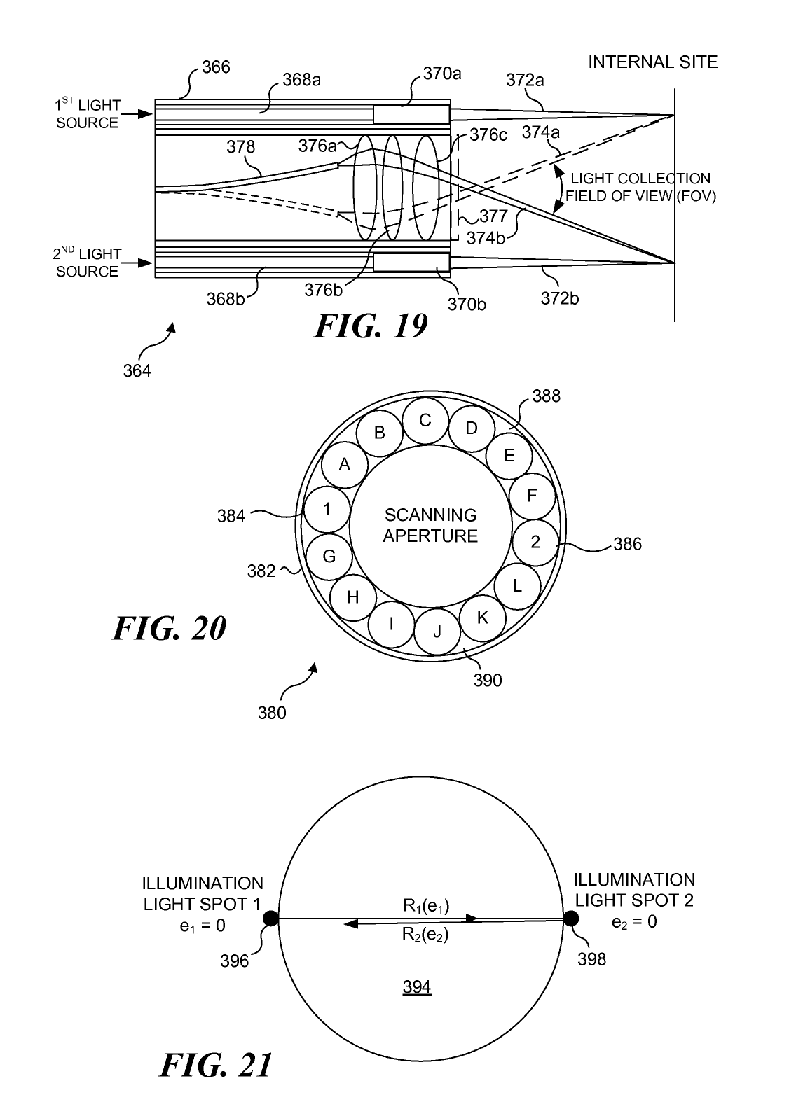

An exemplary SFE probe 364, illustrated in FIG. 19, is configured both for high resolution imaging and higher reflectance light collection, which is useful in measuring and/or mapping local diffuse reflection from a tissue surface at an internal site. Within a housing 366, a first illumination optical fiber 368a conveys illumination light 372a through an optional rod lens 370a, so that the light is focused on an internal site. Similarly, a second illumination optical fiber 368b conveys illumination light 372b through a rod lens 370b, which focuses the light on the internal site. Light 374a and light 374b, corresponding respectively to illumination light 372a and 372b, is reflected from the internal site into lenses 376a, 376b, and 376c, which are disposed adjacent a transparent window 377 and focus the reflected light into the distal end of a scanning optical fiber 378.

In this embodiment, scanning optical fiber 378 is a dual clad, singlemode core optical fiber. For purposes of imaging the internal site, the singlemode core conveys the reflected light to a detector that is disposed at a proximal end of the scanning optical fiber. Optional spatial filtering may be employed in a confocal geometry of illumination and collection through the same singlemode dual clad optical fiber core to selectively collect light from a desired axial depth. However, the inner multimode cladding layer of the scanning optical fiber, which has a substantially great numerical aperture (NA) than the singlemode core, can be employed for higher reflectance light collection (i.e., for collecting more light reflected from the internal site), which can be useful in detecting certain characteristics of the collected light, and thus, evaluating the condition of the tissue from where the light is collected. As one example of how this SFE probe can be used, illumination optical fiber 368a might convey either monochrome light or red, green, and blue light to illuminate the internal site for imaging or for other purposes, while illumination optical fiber 368b might convey polarized light or light of specific wavelength intended for diagnostic purposes. The scanning optical fiber can then sequentially image the internal site with the singlemode core, and then collect the light used for diagnostic or other purposes using the inner cladding layer. The sequence of imaging and collecting light can alternate frame-by-frame, or can be carried out in some other desired sequence.

It should also be understood that an SFE probe can include other optical fibers besides the one or more illumination optical fibers and a scanning optical fiber. FIG. 20 illustrates the distal end face of an exemplary SFE probe 380 that includes a housing 382 with a first illumination optical fiber 384, a second illumination optical fiber 386, six multimode optical fibers 388 (identified individually as “A”-“F”), and six multimode optical fibers 390 (identified individually as “G”-“L”), grouped around a central scanning aperture, behind which is disposed the scanning optical fiber (not specifically shown in this Figure). To separate completely diffuse light reflected from the tissue from surface reflections and light scattered from the top surfaces, polarization filtering may be employed. To select light that backscatters from deeper regions of the internal site, cross-polarization filtering can be used. To implement this approach, the illumination fibers would be polarization preserving optical fibers of a specific orientation. However, in contrast to FIG. 17, the polarization filter used in FIG. 19 would be disposed centrally, filtering photons striking the resonant scanned optical fiber only, and the polarization filter will be orthogonal or cross axis with respect to the illumination polarization axis. One or more of the plurality of multimode optical fibers 388 and 390 can be employed for high-resolution SFE imaging (collecting red, green, and blue backscatter from illumination from the singlemode core of dual clad scanning optical fiber 378), or can be used for conveying additional illumination light, or for delivery of optical therapy, or for light collection useful for diagnostic and other purposes. From this example, it will be evident that many other combinations of one or more illumination optical fibers, a scanning optical fiber and one or more other multimode optical fibers can be provided on almost any of the SFE probes discussed herein, to achieve the benefits of using a scanning device (e.g., a scanning optical fiber or scanning mirror) with one or more separate fixed or non-resonantly movable illumination optical fiber(s).

FIG. 21 illustrates how two or more illumination optical fibers that are coupled to two or more illumination light sources having different characteristics can be employed to achieve differing results when the light from an internal site 394 is received by a scanning optical fiber in an SFE probe like those discussed above. In this Figure, an axial view (i.e., perpendicular to the longitudinal axis of the SFE probe, which is not shown) of the internal site is illustrated. An illumination light spot 396 is produced on internal site 394 by one or more illumination optical fibers of the SFE probe, using a first illumination light source. Similarly, an illumination light spot 398 is produced by one or more other illumination optical fibers on the SFE probe. The circle shown in this Figure represents the FOV of the scanning optical fiber. It should be understood that light spots 396 and 398 can be within or proximal to the light collection area represented by this FOV, and that there can be only one light spot or more than two light spots. Further, the plural light spots can each have different characteristics, or can have the same characteristics, or combinations of lights spots can have different characteristics than other combinations of light spots. The illumination light sources used to produce the different illumination light spots can, for example, produce illumination light of different wavelengths, polarization, coherence, or modulation characteristics. The two vectors shown within the FOV in FIG. 21 illustrate that the light used to produce light spot 396 has different characteristics than that used to produce light spot 398. If an illumination optical fiber deflector is used, then the two light spots shown at the periphery of the imaging field can be brought together at a single point within the field, ideally to form a central point source illumination. The diffuse reflectance R(ρ) from the tissue surface can be measured using a resonant spiral or slowly varying circular scan pattern, which enables highly accurate measurement of local optical properties of the illuminated tissue.

### Exemplary Scanning System

FIG. 22 illustrates a system 450 that shows how the signals produced by an SFE probe that is inside a patient's body are processed with external instrumentation, and how signals used for controlling the SFE probe system to vary the scanning parameter(s) in scanning frames are input to the SFE probe that is positioned inside the patient's body. In order to provide integrated imaging and other functionality, system 450 is thus divided into the components that remain external to the patient's body, and those which are used internally (i.e., the components on the SFE probe within a dash line 452—some of which are optional). A block 454 lists the functional components disposed at the distal end of the SFE system. As indicated therein, these exemplary components can include illumination optics, one or more electromechanical scan actuator(s) that can drive the scanning optical fiber or scanning mirror, one or more illumination optical fiber actuator(s), optional photon detectors for imaging the internal site, and optionally, additional photon detectors or multimode optical fibers for diagnostic purposes and for therapy and/or monitoring purposes. The photon collectors for imaging can be discrete sensors mounted on the SFE probe, or may be separate multimode optical fibers, such as those shown in FIG. 20, discussed above. It should be noted that in regard to system 450, only the functional components actually required for a specific application of the SFE system may be included. Also, additional functions besides imaging can be diagnostic, or therapy, or any combination thereof.

Externally, the illumination optics and scanner(s) are supplied light from imaging sources and modulators, as shown in a block 456. Further details concerning several preferred embodiments of external light source systems 458 for producing RGB, UV, IR, and high-intensity light conveyed to the distal end of an optical fiber system will be evident to a person of ordinary skill in this art. Scanner sensors (optional) can be used to produce a signal that is fed back to control the scanner actuators, illumination source, and modulators, to implement scanning control after signal processing in a block 460.

In block 460, image signal filtering, buffering, scan conversion, amplification, and other processing functions are implemented using the electronic signals produced by the imaging photon detectors and any other photon detectors employed for diagnosis/therapy, and monitoring purposes. Blocks 456, 458, and 460 are interconnected bi-directionally to convey signals that facilitate the functions performed by each respective block. Similarly, each of these blocks is bi-directionally coupled in communication with a block 462 in which analog-to-digital (A/D) and digital-to-analog (D/A) converters are provided for processing signals that are supplied to a computer workstation user interface or other computing device, which can be employed for image acquisition, processing, for executing related programs, and for other functions. Control signals from the computer workstation are fed back to block 462 and converted into analog signals, where appropriate, for controlling or effecting each of the functions provided in blocks 456, 458, and 460. The A/D converters and D/A converters within block 462 are also coupled bi-directionally to a block 464 in which data storage is provided, and to a block 466. Block 466 represents a user interface for maneuvering, positioning, and stabilizing the SFE probe within a patient's body.

In block 464, the data storage is used for storing the image data produced by the detectors within a patient's body, and for storing other data related to the imaging and functions implemented by the SFE probe. Block 464 is also coupled bi-directionally to a computer workstation 468 and to interactive display monitor(s) in a block 470. Block 470 receives an input from block 460, enabling images of the internal site to be displayed interactively. In addition, one or more passive video display monitors may be included within the system, as indicated in a block 472. Other types of display devices 474, for example, a head-mounted display (HMD) system, can also be provided, enabling medical personnel to view the internal site as a pseudo-stereo image.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. An optical fiber system that is selectively operable in a plurality of different modes, for illuminating an internal site within a body of a patient with different types of light for each of at least two different modes, and responsive to light received from the internal site in at least one mode of the plurality of different modes, comprising:
(a) a plurality of light sources that produce the different types of light, from which at least one light source can be selectively energized during operation of the optical fiber system in a current mode;
(b) an illumination optical fiber having a distal end, the illumination optical fiber being selectively coupled to different ones of the plurality of light sources to convey the type of light emitted thereby to the distal end of the illumination optical fiber during operation in the current mode, to illuminate the internal site with one of the plurality of different types of light;
(c) a scanning driver that is adapted to be energized by a drive signal;
(d) a scanner optical fiber having a proximal end, and a distal end that is connected to the scanning driver, the scanning driver being configured to scan at least a portion of the internal site in a desired pattern, to receive light from the at least the portion of the internal site that has been illuminated by light from the illumination optical fiber in the current mode, the light that is received by the scanner optical fiber entering the distal end of the scanner optical fiber and being conveyed by the scanner optical fiber toward its proximal end; and
(e) a light sensor coupled to the scanner optical fiber to receive the light being conveyed by the scanner optical fiber, to produce an output signal indicative of at least one parameter of the light received from the at least the portion of the internal site when the optical fiber scanning system is operating in the current mode.

2. The optical fiber scanning system of claim 1, wherein the at least two different modes are selected from the group consisting of:
(a) a diagnostic mode in which a diagnostic light is conveyed through the illumination optical fiber;
(b) an imaging mode in which an imaging light is conveyed through the illumination optical fiber; and
(c) a therapy mode in which a relatively high-power therapy light is conveyed through the illumination optical fiber.

3. The optical fiber scanning system of claim 1, further comprising a display coupled to the light sensor to receive the output signal from the light sensor, for producing an image of the at least the portion of the internal site, in response to the output signal.

4. The optical fiber scanning system of claim 1, wherein the light received from the at least the portion of the internal site entering the distal end of the scanner optical fiber is at a substantially different wavelength than the light produced by one of the plurality of light sources that is conveyed through the illumination optical fiber to illuminate the internal site in the current mode of operation.

5. The optical fiber scanning system of claim 1, wherein the at least one parameter comprises at least one of:
(a) an intensity of the light received;
(b) a direction along which the light received traveled from the internal site;
(c) an angle along which the light received traveled from the internal site;
(d) a distance along which the light received traveled from the internal site;
(e) a period of time during which the light received traveled from the internal site;
(f) a depth within tissue at the internal site from which the light received traveled; and
(g) a wavelength of the light received.

6. The optical fiber scanning system of claim 1, further comprising an illumination optical fiber displacer that is coupled to the illumination optical fiber and which is configured to selectively displace the illumination optical fiber with a non-resonant motion, so that the illumination optical fiber illuminates a different region of the internal site than when the illumination optical fiber was at a previous position.

7. The optical fiber scanning system of claim 6, wherein the illumination optical fiber displacer changes a direction in which the illumination optical fiber emits one of the plurality of different types of light directed toward the internal site, to illuminate the different region with said one of the plurality of different types of light in the current mode.

8. The optical fiber scanning system of claim 6, wherein the illumination optical fiber displacer changes a depth of focus of said one of the plurality of different types of light emitted by the illumination optical fiber, within tissue at the internal site, to illuminate the different region at a different depth within the tissue.

9. The optical fiber scanning system of claim 6, wherein the illumination optical fiber displacer comprises a cable that is moved relative to the scanner optical fiber, to displace the distal end of the illumination optical fiber.

10. The optical fiber scanning system of claim 6, wherein the illumination optical fiber displacer comprises an elastomeric membrane defining a volume that is coupled in fluid communication with a source of pressurized fluid and which is disposed adjacent to the distal end of the illumination optical fiber so as to displace said distal end when an amount of pressurized fluid within the volume is selectively changed.

11. The optical fiber scanning system of claim 6, wherein the illumination optical fiber displacer comprises an electromechanical actuator disposed adjacent to the distal end of the illumination optical fiber so as to displace said distal end in response to an electrical signal that is selectively applied thereto.

12. The optical fiber scanning system of claim 6, wherein the illumination optical fiber displacer comprises a wire and a restoring spring actuator, and is disposed adjacent to the distal end of the illumination optical fiber so as to displace said distal end when a force is selectively applied to the wire.

13. The optical fiber scanning system of claim 1, wherein the scanning driver drives the distal end of the scanner optical fiber to move in the desired pattern at approximately a resonant frequency of the distal end of the scanner optical fiber.

14. The optical fiber scanning system of claim 1, further comprising a movable reflective surface disposed to reflect the light received traveling from the at least the portion of the internal site toward the distal end of the scanner optical fiber, wherein the movable reflective surface is driven to move at approximately a resonant frequency by the scanning driver, to thereby scan the at least the portion of the internal site in the desired pattern.

15. The optical fiber scanning system of claim 1, further comprising a processor that measures a position of reflected light within an image field of view originating from a position and orientation of the illumination optical fiber within the optical fiber scanning system, in regard to at least a portion of the internal site that is causing a reflection of one of the plurality of different types of light.

16. The optical fiber scanning system of claim 1, wherein the illumination optical fiber comprises a multimode optical fiber.

17. The optical fiber scanning system of claim 1, wherein the illumination optical fiber comprises a singlemode optical fiber.

18. The optical fiber scanning system of claim 1, wherein the scanner optical fiber comprises an optical fiber selected from the group consisting of:
(a) a multimode optical fiber;
(b) a singlemode optical fiber;
(c) a multi-clad optical fiber; and
(d) both a multimode and a singlemode optical fiber.

19. The optical fiber scanning system of claim 1, further comprising a processor for determining particulate size in tissue at the at least the portion of the internal site, as a function of a scattering angle of the light received by the scanner optical fiber, wherein the particulate size is proportional to an intensity of the light received and the scattering angle of the light received from particulates comprising the tissue at the internal site.

20. The optical fiber scanning system of claim 19, wherein the particulates are cellular components.

21. The optical fiber scanning system of claim 1, wherein the desired pattern for scanning is a spiral, further comprising a processor for determining a characteristic of tissue at the internal site, wherein the characteristic is a reflectance measured as a function of a distance from a point where the tissue is illuminated by one of the plurality of different types of light, the reflectance being used to determine one parameter selected from the group consisting of: an absorption, a scattering, and an effective attenuation coefficient of the tissue that is illuminated.

22. The optical fiber scanning system of claim 1, wherein the desired pattern for scanning is a spiral, further comprising a processor for determining a characteristic of the tissue at the internal site, wherein the characteristic is a reflectance measured as a function of a propagation time for light from a point where the tissue is illuminated, the propagation time being used by the processor to determine a parameter selected from the group consisting of: an absorption, a scattering, and an effective attenuation coefficient of the tissue that is illuminated by the light.

23. The optical fiber scanning system of claim 1, wherein the desired pattern for scanning is selected from the group consisting of:
(a) a spiral scan;
(b) a propeller scan;
(c) a linear scan;
(d) a raster scan; and
(e) a Lissajous scan.

24. The optical fiber scanning system of claim 1, wherein the at least one parameter comprises at least one of one of a distance and an angle between the optical fiber scanning system and a surface of the tissue at the internal site, and wherein the at least one parameter is determined in response to a specular reflection from a surface of the tissue at the internal site detected in the light received.

25. The optical fiber scanning system of claim 1, wherein a light source from the plurality of light sources that is coupled to the illumination fiber in the current mode, produces light having at least one characteristic selected to render a therapy to the internal site, wherein the characteristic comprises at least one selected from the group consisting of a power level, and a waveband of the light.

26. The optical fiber scanning system of claim 1, further comprising a lens disposed proximate to the distal end of the illumination optical fiber for focusing the light directed toward the internal site in the current mode.

27. A method for scanning an internal site within a patient's body in a plurality of different modes, wherein for each of at least two modes of the plurality of different modes that is implemented, the method comprises the steps of:
(a) conveying one of a plurality of different types of light from a source selected for use in a current mode of the at least two modes, toward a distal end of an illumination optical fiber, and directing the light emitted from the distal end onto the internal site, wherein the distal end of the illumination optical fiber is stationary or relatively slowly movable with a non-resonant motion;
(b) scanning at least a portion of the internal site to collect received light from the at least the portion of the internal site;
(c) conveying the received light from the at least the portion of the internal site through a scanner optical fiber and toward a proximal end of the scanner optical fiber;
(d) detecting the received light and producing an output signal in response to the received light; and
(e) using the output signal to determine at least one parameter of the at least the portion of the internal site, for the current mode that is then being implemented.

28. The method of claim 27, wherein the at least two modes are selected from the group consisting of:
(a) a diagnostic mode in which a diagnostic light is conveyed through the illumination optical fiber;
(b) an imaging mode in which an imaging light is conveyed through the illumination optical fiber; and
(c) a therapy mode in which a relatively high-power therapy light is conveyed through the illumination optical fiber.

29. The method of claim 27, wherein the step of using the output signal comprises the step of displaying an image of the at least the portion of the internal site, in response to the output signal, the at least one parameter comprising a visual appearance of the at least the portion of the internal site.

30. The method of claim 27, wherein the received light is at a substantially different wavelength than the light that is directed at the internal site from the illumination optical fiber, and wherein the step of using the output signal comprises the step of determining a characteristic of tissue at the internal site.

31. The method of claim 27, wherein the at least one parameter is selected from the group consisting of:
(a) an intensity of the received light;
(b) a power of the received light;
(c) a direction along which the received light traveled;
(d) an angle along which the received light traveled from the internal site;
(e) a depth within tissue at the internal site from which the received light traveled;
(f) a wavelength of the received light; and
(g) a propagation time of the received light.

32. The method of claim 27, further comprising the step of selectively displacing the illumination optical fiber so that it illuminates a different region of the internal site.

33. The method of claim 32, wherein the step of selectively displacing comprises the step of changing a direction in which the illumination optical fiber emits light directed toward the internal site, to illuminate the different region.

34. The method of claim 32, wherein the step of selectively displacing comprises the step of changing a depth of focus of the light emitted by the illumination optical fiber within tissue at the internal site, to illuminate the different region at a different depth within the tissue.

35. The method of claim 27, wherein the step of scanning comprises the step of driving the distal end of the scanner optical fiber to move at approximately a resonant frequency of the distal end of the scanner optical fiber.

36. The method of claim 27, wherein the step of scanning comprises the step of driving a movable reflective surface disposed to reflect the received light from the at least the portion of the internal site toward the distal end of the scanner optical fiber, so that the movable reflective surface is driven to move at approximately a resonant frequency of the movable reflective surface.

37. The method of claim 27, wherein the step of using the output signal comprises the step of determining a position of a reflection of the light illuminating the tissue within a field of view of the received light collected by the scanner optical fiber

38. The method of claim 27, wherein the step of using the output signal comprises the step of determining a particulate size in tissue at the internal site, as a function of a scattering angle of the received light, wherein the particulate size is proportional to an intensity of the received light and the scattering angle of the received light.

39. The method of claim 38, wherein the step of determining a particulate size comprises the step of determining the size of cellular components in the tissue.

40. The method of claim 27, wherein the desired pattern for scanning is a spiral, wherein the step of using the output signal comprises the step of determining a characteristic of tissue at the internal site, and wherein the characteristic is a reflectance measured as a function of a distance from a point where light emitted from the illumination optical fiber is incident on the tissue, the reflectance being used to determine a parameter selected from the group consisting of: an absorption, a scattering, and an effective attenuation coefficient of the tissue that is illuminated by the light.

41. The method of claim 27, wherein the step of scanning comprises the step of scanning with a pattern selected from the group consisting of:
(a) a spiral pattern;
(b) a propeller pattern;
(c) a linear pattern;
(d) a raster pattern; and
(e) a Lissajous pattern.

42. The method of claim 27, wherein the step of using the output signal comprises the step of determining at least one parameter selected from the group consisting of: a distance and an angle between the optical fiber scanning system and a surface of the tissue at the internal site in response to a specular reflection from the surface of the tissue that is included in the received light.

43. The method of claim 27, wherein when operating in a therapy mode, further comprising the step of emitting light from the illumination optical fiber having at least one characteristic selected for rendering a therapy to the internal site, wherein the at least one characteristic comprises at least one selected from the group consisting of: a relatively high-power, and a waveband of said light.

44. The method of claim 27, further comprising the step of focusing the light directed toward the internal site from the illumination optical fiber.

45. An optical fiber scope for illuminating an internal site within a body of a patient with a plurality of different types of light during a plurality of different modes, and responding to light received from the internal site, comprising:
(a) a plurality of different light sources producing different types of light for illuminating the internal site;
(b) an elongate housing disposed at a distal end of the optical fiber scope;
(c) a scanner disposed generally centrally at the distal end of the optical fiber scope, the scanner being driven to move in a desired pattern at approximately a resonant frequency and configured so that received light from at least a desired portion of the internal site is collected by the scanner and conveyed through a scanner optical fiber toward a proximal end of the scanner optical fiber;
(d) a plurality of illuminating optical fibers having distal ends that are spaced apart and disposed around the scanner, within the elongate housing, the plurality of illuminating optical fibers conveying light from a selected one of the plurality of different light sources toward the distal ends of the illuminating optical fibers during operation in a current mode, so that the light emitted from the distal ends of the illuminating optical fibers is directed to the internal site; and
(e) a sensor coupled to the scanner optical fiber and responsive to the received light that is conveyed through the scanner optical fiber, the sensor producing an output signal that can be processed to provide data relating to tissue at the internal site for the current mode that is being implemented by the optical fiber scope.

46. The optical fiber scope of claim 45, wherein the at least two different modes are selected from the group consisting of:
(a) a diagnostic mode in which a diagnostic light is conveyed through the plurality of illumination optical fibers;
(b) an imaging mode in which an imaging light is conveyed through the plurality of illumination optical fibers; and
(c) a therapy mode in which a relatively high-power therapy light is conveyed through the plurality of illumination optical fibers.

47. The optical fiber scope of claim 45, wherein the scanner comprises a driver coupled to the distal end of the scanner optical fiber, the driver applying force that resonantly moves the distal end in the desired pattern, to selectively scan the desired portion of the internal site.

48. The optical fiber scope of claim 45, further comprising a displacer for selectively displacing the distal ends of the illuminating optical fibers to control where light emitted from the illuminating optical fibers is incident on the internal site.

49. The optical fiber scope of claim 45, wherein the sensor is responsive to the received light of a specific waveband that is substantially different than that of the light emitted by the plurality of illuminating optical fibers during operation in the current mode.

50. The optical fiber scope of claim 45, wherein the scanner comprises a reflective surface that reflects the received light into the distal end of the scanner optical fiber and includes a driver for moving the reflective surface at approximately a resonant frequency.

51. The optical fiber scope of claim 45, wherein the scanner comprises an optical fiber that transmits the received light, and wherein the optical fiber is of a type that is selected from the group consisting of:
(a) a multimode optical fiber;
(b) a singlemode optical fiber;
(c) a multi-clad optical fiber; and
(d) a combined multimode and singlemode optical fiber.

52. The optical fiber scope of claim 45, wherein the scanner comprises a processor that measures a position of reflected light within an image field of view resulting from a position and an orientation of the plurality of illumination optical fibers within the optical fiber scanning system, and in regard to at least a portion of the internal site from which the received light is reflected.

53. The optical fiber scope of claim 45, wherein the desired pattern for scanning comprises a pattern selected from the group consisting of:
(a) a spiral pattern;
(b) a propeller pattern;
(c) a linear pattern;
(d) a raster pattern; and
(e) a Lissajous pattern.

54. The optical fiber scope of claim 45, wherein the output signal produced by the sensor is usable for displaying an image of the at least the portion of the internal site in both an imaging mode and a monitoring mode.

55. The optical fiber scope of claim 45, wherein the output signal produced by the sensor is usable for calculating an absorption relative to a depth of penetration of the light emitted from the plurality of illuminating optical fibers.

56. The optical fiber scope of claim 45, wherein the output signal produced by the sensor is usable for determining at least one parameter selected from the group consisting of a distance and an angle between the optical fiber scope and a surface of the tissue at the internal site, in response to a specular reflection from the surface of the tissue, the specular reflection comprising the received light.

57. The optical fiber scope of claim 45, wherein the output signal produced by the sensor is usable for determining a particulate size in the tissue at the internal site, as a function of a scattering angle of the received light, and wherein the particulate size is proportional to an intensity of the received light and the scattering angle of the received light.

58. The optical fiber scope of claim 57, wherein the particulate size corresponds to a size of a cellular component.

59. The optical fiber scope of claim 45, wherein the output signal produced by the sensor is usable for calculating a parameter selected from the group consisting of: an absorption, a scattering, and an effective attenuation coefficient of the tissue at the internal site, as a function of a reflectance measured from a point where the tissue is illuminated by the light emitted from the plurality of illuminating optical fibers in the current mode.
